(19) United States
(12) Patent Application Publication
(10) Pub. No.: US 2023/0255078 A1
(43) Pub. Date: Aug. 10, 2023

(54) Light Emitting Display Device

(71) Applicant: LG Display Co., Ltd., Seoul (KR)

(72) Inventors: Wook Song, Paju-si (KR); Yu Jeong Lee, Paju-si (KR); Byung Gun Ahn, Paju-si (KR); Mi Young Han, Paju-si (KR); Chun Ki Kim, Paju-si (KR); Sae Mi Park, Paju-si (KR); Jung Keun Kim, Paju-si (KR); Byung Soo Kim, Paju-si (KR)

(21) Appl. No.: 18/078,886

(22) Filed: Dec. 9, 2022

(30) Foreign Application Priority Data
Dec. 31, 2021 (KR) 10-2021-0194802

(51) Int. Cl. H10K 59/35; H10K 50/13; H10K 50/17; H10K 50/16; H10K 59/38; C07D 401/14; C07D 209/08

(52) U.S. Cl. CPC H01L 27/3213; C07D 209/08; C07D 401/14; H01L 27/322; H01L 51/504; H01L 51/5072; H01L 51/5092

(57) ABSTRACT

A light emitting device is capable of improving the efficiency in the visible light wavelength in a structure in which light extraction efficiency is improved through a microlens array. The light emitting display device includes a substrate having a plurality of subpixels, each comprising a light emitting region and a non-light emitting region, an over-coating layer having irregularities on a surface thereof in at least the light emitting region, and a light emitting device disposed on the surface of the over-coating layer, the light emitting device comprising an anode and a cathode facing each other, and at least two blue stacks and a phosphorescent stack between the anode and the cathode, at least one of the blue stacks is the blue light emitting layer and includes an electron transport layer including a first material, the electron transport layer being in contact with the blue light emitting layer.

### CROSS-REFERENCE TO RELATED APPLICATIONS

This application claims the benefit of Republic of Korea Patent Application No. 10-2021-0194802, filed on Dec. 31, 2021, which is hereby incorporated by reference as if fully set forth herein.

### BACKGROUND

### Field

The present disclosure relates to a light emitting display device, and more particularly to a light emitting display device that is capable of similarly increasing efficiency in all of blue, green, and red wavelengths in a structure in which a microlens is provided in a light emitting region of a light emitting device including a plurality of stacks.

### Discussion of the Related Art

Recently, a light emitting display device that does not require a separate light source and has a light emitting device in a display panel without a separate light source to make the display device compact and realize clear color has been considered a competitive application.

Meanwhile, the light emitting device currently used in a light emitting display device requires higher efficiency in order to realize a desired image quality, and is preferably implemented in the form of a plurality of stacks.

In addition, in recent years, a method in which a microlens array is provided in a light emitting region in order to emit the light intrinsically emitted from a light emitting device to the outside with increased efficiency has been considered.

### SUMMARY

Accordingly, the present disclosure is directed to a light emitting display device that substantially obviates one or more problems due to the limitations and disadvantages of the related art.

A microlens array may be provided as a pattern having structural irregularities on a surface that is in contact with a light emitting device. However, the light emitting device includes a plurality of light emitting layers within a distance within which the thickness of organic layers in an anode and a cathode is less than 1 µm, and emits light through repeated resonance based on reflection and re-reflection by different light emitting layers. It is difficult to control all colors with the same efficiency through the microlens array.

In particular, when the microlens array is used in a light emitting device in which a blue stack having a blue light emitting layer using a fluorescent dopant and a phosphorescent stack having a phosphorescent layer using a phosphorescent dopant are laminated in a tandem manner, only the efficiency of emission of light of long-wavelength colors is improved, without improvement in the efficiency of emission of blue light. As a result, the efficiency of emission of blue light from the light emitting display device may be relatively low after a final color filter is applied.

Additional advantages, objects, and features of the invention will be set forth in part in the description which follows and in part will become apparent to those having ordinary skill in the art upon examination of the following, or may be learned from practice of the invention. The objectives and other advantages of the invention may be realized and attained by the structure particularly pointed out in the written description and claims hereof as well as the appended drawings.

To achieve these objects and other advantages and in accordance with the purpose of the disclosure, as embodied and broadly described herein, a light emitting display device includes a substrate having a plurality of subpixels, each including a light emitting region and a non-light emitting region, an over-coating layer having irregularities on a surface thereof in at least the light emitting region, and a light emitting device disposed on the surface of the over-coating layer, the light emitting device including an anode and a cathode facing each other, and at least two blue stacks and a phosphorescent stack between the anode and the cathode, wherein each of the blue stacks includes a blue light emitting layer having an emission peak of 420 nm to 480 nm, at least one of the blue stacks the blue light emitting layer and includes an electron transport layer including a first material of Formula 1, the electron transport layer being in contact with the blue light emitting layer, and the phosphorescent stack includes at least two phosphorescent light emitting layers emitting light having a wavelength longer than that of the blue light emitting layer.

In another aspect, a light emitting display device includes a substrate having a plurality of subpixels, each comprising a light emitting region and a non-light emitting region, an over-coating layer having a plurality of concave portions and convex portions on a surface of the over-coating layer in a light emitting region for a subpixel, and a white light emitting device disposed on the surface of the over-coating layer for the subpixel. The white light emitting device further includes an anode, an organic layer on the anode, and a cathode on the organic layer, wherein the anode, the organic layer, and the cathode have a plurality of concave portions and convex portions corresponding to the concave portions and the convex portions of the over-coating layer respectively. The organic layer further includes one or more blue stacks for emitting blue light wherein at least a first blue stack comprises an electron transport layer including a first material of Formula 1, and one or more phosphorescent stacks for emitting light at a higher wavelength than the one or more blue stacks.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the disclosure and are incorporated in and constitute a part of this application, illustrate embodiment(s) of the invention and together with the description serve to explain the principle of the disclosure.

FIG. 1 is a plan view illustrating an arrangement of subpixels according to an embodiment of the present disclosure.

FIG. 2 is a cross-sectional view illustrating a single subpixel of FIG. 1 according to an embodiment of the present disclosure.

FIG. 3A is an optical image illustrating an example of a light emitting region of a white subpixel of FIG. 1 according to an embodiment of the present disclosure.

FIG. 3B is an enlarged view of the cross section of FIG. 3A according to an embodiment of the present disclosure.

FIG. 4 is a cross-sectional view illustrating the light emitting device shown in FIG. 2, used in first to third experimental example groups according to an embodiment of the present disclosure.

FIG. 5 shows white color coordinates expressed by a light emitting display device including light emitting devices used in third to fifth experimental examples.

FIG. 6 is a graph showing emission spectra of the light emitting devices of third to fifth experimental examples.

FIG. 7 is a graph showing white efficiency and module efficiency of light emitting display devices using the light emitting devices of the third to fifth experimental examples.

### DETAILED DESCRIPTION

Reference will now be made in detail to preferred embodiments of the present invention, examples of which are illustrated in the accompanying drawings. Wherever possible, the same reference numbers will be used throughout the drawings to refer to the same or like parts. In the following description of the present invention, detailed descriptions of known functions and configurations incorporated herein will be omitted when the same may obscure the subject matter of the present invention. In addition, the names of elements used in the following description are selected in consideration of clarity of description of the specification, and may differ from the names of elements of actual products.

The shape, size, ratio, angle, number, and the like shown in the drawings to illustrate various embodiments of the present invention are merely provided for illustration, and the invention is not limited to the content shown in the drawings. Wherever possible, the same reference numbers will be used throughout the drawings to refer to the same or like parts. In the following description, detailed descriptions of technologies or configurations related to the present invention may be omitted so as to avoid unnecessarily obscuring the subject matter of the present invention. When terms such as “including”, “having”, and “comprising” are used throughout the specification, an additional component may be present, unless “only” is used. A component described in a singular form encompasses a plurality thereof unless particularly stated otherwise.

The components included in the embodiments of the present invention should be interpreted to include an error range, even if there is no additional particular description thereof.

In describing the variety of embodiments of the present invention, when terms describing positional relationships such as “on”, “above”, “under” and “next to” are used, at least one intervening element may be present between the two elements, unless “immediately” or “directly” is also used.

In describing the variety of embodiments of the present invention, when terms related to temporal relationships, such as “after”, “subsequently”, “next”, and “before”, are used, the non-continuous case may be included, unless “immediately” or “directly” is also used.

In describing the variety of embodiments of the present invention, terms such as “first” and “second” may be used to describe a variety of components, but these terms only aim to distinguish the same or similar components from one another. Accordingly, throughout the specification, a “first” component may be the same as a “second” component within the technical concept of the present invention, unless specifically mentioned otherwise.

Features of various embodiments of the present disclosure may be partially or completely coupled to or combined with each other, and may be variously inter-operated with each other and driven technically. The embodiments of the present disclosure may be carried out independently from each other, or may be carried out together in an interrelated manner.

As used herein, the term “doped” means that, in a material that accounts for most of the weight of a layer, a material (for example, n-type and p-type materials, or organic and inorganic substances) having physical properties different from the material that occupies most of the weight ratio of the layer is added in an amount less than 30% by weight. In other words, a “doped” layer is a layer that is used to distinguish a host material from a dopant material of a certain layer, in consideration of the weight ratio. Also, the term “undoped” refers to any case other than the “doped” case. For example, when a layer contains a single material or a mixture of materials having the same properties as each other, the layer is included in the “undoped” layer. For example, if at least one of the materials constituting a certain layer is p-type and not all materials constituting the layer are n-type, the layer is included in the “undoped” layer. For example, if at least one of materials constituting a layer is an organic material and not all materials constituting the layer are inorganic materials, the layer is included in the “undoped” layer. For example, if all materials constituting a certain layer are organic materials, at least one of the materials constituting the layer is n-type and the other is p-type, when the n-type material is present in an amount of less than 30 wt%, or when the p-type material is present in an amount of less than 30 wt%, the layer is included in the “doped” layer.

Hereinafter, a light emitting display device according to the present disclosure will be described with reference to the drawings.

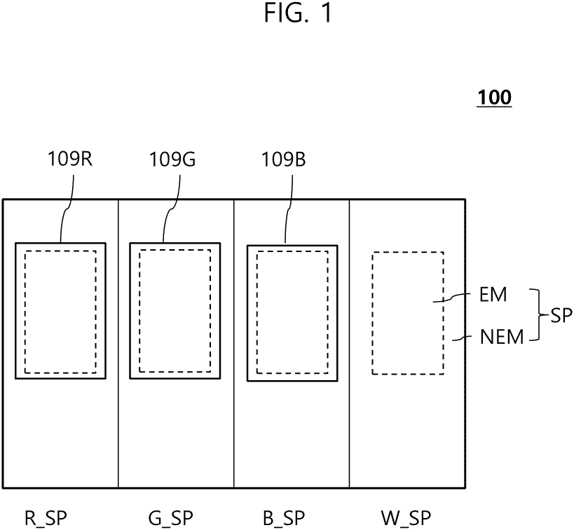

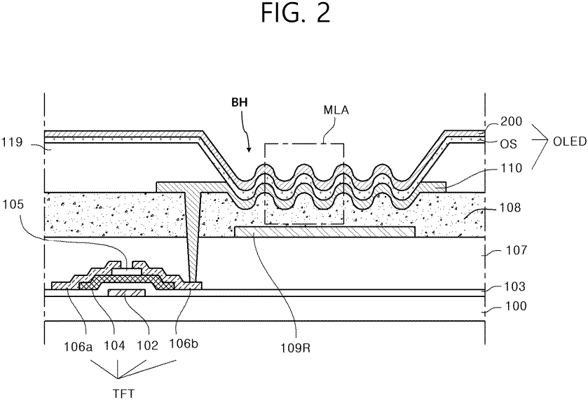

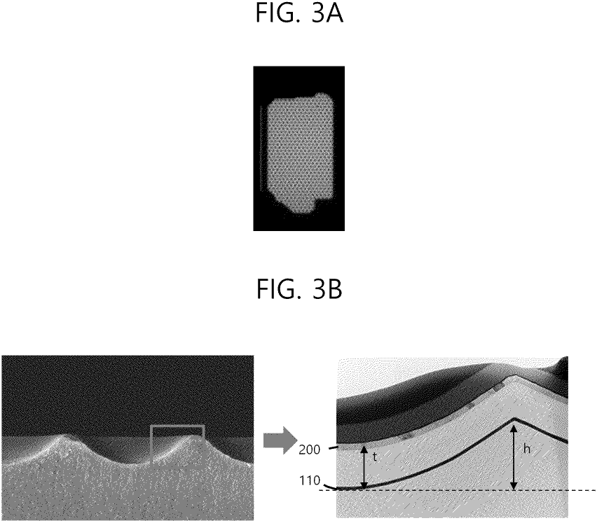

FIG. 1 is a plan view illustrating an arrangement of subpixels according to an embodiment of the present disclosure, and FIG. 2 is a cross-sectional view illustrating a single subpixel of FIG. 1 according to an embodiment of the present disclosure. FIG. 3A is an optical image illustrating an example of a light emitting region of a white subpixel of FIG. 1 and FIG. 3B is an enlarged view of the cross section of FIG. 3A according to an embodiment of the present disclosure.

As shown in FIGS. 1 and 2, the light emitting display device of the present disclosure includes a substrate 100 having a plurality of subpixels R_SP, G_SP, B_SP, and W_SP, a light emitting device (also referred to as an “OLED”, or “organic light emitting diode”) commonly provided on the substrate 100, a thin film transistor (TFT) provided in each of the subpixels and connected to an anode 110 of the light emitting device (OLED), and a color filter layer 109R, 109G, or 109B provided below the anode 110 of at least one of the subpixels.

The illustrated example relates to a configuration including the white subpixel W_SP, but the present disclosure is not limited thereto. A configuration in which the white subpixel W_SP is omitted and only the red, green, and blue subpixels R_SP, G_SP, and B_SP are provided is also possible. In some cases, a combination of a cyan subpixel, a magenta subpixel, and a yellow subpixel capable of creating white may be used, instead of the red, green, and blue subpixels. Each of the subpixels R_SP, G_SP, B_SP, and W_SP may have a light emitting region EM that emits light and a non-light emitting region NEM around the light emitting region EM. The non-light emitting region NEM includes a bank 119 to divide the region. The non-light emitting region NEM may include lines such as gate lines, data lines, driving power voltage lines, and reference power supply voltage lines, and pixel-driving circuits such as driving thin film transistors, switching thin film transistors, and storage capacitors.

The thin film transistor TFT constituting the driving thin film transistor and the switching thin film transistor includes, for example, a gate electrode 102, a semiconductor layer 104, and a source electrode 106a and a drain electrode 106b connected to both surfaces of the semiconductor layer 104. In addition, a channel passivation layer 105 may be further provided on the portion where the channel of the semiconductor layer 104 is disposed in order to prevent direct connection between the source/drain electrodes 106a and 106b and the semiconductor layer 104.

A gate insulating layer 103 is provided between the gate electrode 102 and the semiconductor layer 104. The gate insulating layer 103 may be an inorganic layer such as an oxide layer, a nitride layer, or an oxynitride layer.

The semiconductor layer 104 may be formed of, for example, an oxide semiconductor, amorphous silicon, polycrystalline silicon, or a combination thereof. For example, when the semiconductor layer 104 is formed of an oxide semiconductor, the heating temperature required for forming the thin film transistor can be lowered and thus the substrate 100 can be selected from a greater variety of available types thereof, so the semiconductor layer 104 can be advantageously applied to a flexible display device.

In addition, the drain electrode 106b of the thin film transistor TFT may be connected to the anode 110 through a contact hole CT provided in a passivation layer 107 and an over-coating layer 108.

The passivation layer 107 is provided to primarily protect the thin film transistor TFT, and color filters 109R, 109G, and 109B may be provided thereon. The passivation layer 107 may be either an inorganic insulating film or an organic insulating film, or a hybrid film formed of a combination of an inorganic component with an organic component.

When the plurality of subpixels includes a red subpixel, a green subpixel, a blue subpixel, and a white subpixel, the color filter may include first to third color filters in each of the remaining subpixels 109R, 109G, and 109B, excluding the white subpixel W_SP, and may allow the emitted white light to pass through the anode 110 for each wavelength. The second over-coating layer 108 is formed under the anode 110 to cover the first to third color filters 109R, 109G, and 109B.

As shown in FIG. 2, the over-coating layer 108 has irregularities on the surface thereof. The irregularities may be formed by a reflow process over the entire surface of the passivation layer 107 using an organic material in a region corresponding to at least the light emitting region of each subpixel. The surface of the overcoat layer 108 is also referred to as a “microlens array” in that the over-coating layer 108 has a lens shape, and the width and height of each irregularity unit are in micro units. In one embodiment, the over-coating layer 108 has a plurality of concave portions and a plurality of convex portions on the surface.

Meanwhile, the anode 110 is formed on the surface of the overcoat layer 108 excluding the contact hole CT, and is provided along the irregularities of the surface of the over-coating layer 108 described above.

In addition, the light emitting device OLED includes the anode 110, the cathode 200, and an organic layer OS in the anode 110 and the cathode 200. The organic layer OS has a total thickness of less than 1 µm, although it includes a plurality of stacks and both the anode 110 and the cathode 200 are formed along the surface irregularities of the over-coating layer 108. Thus, the anode 110, the cathode 200, and the organic layer OS may have a plurality of concave portions and a plurality of convex portions corresponding to the concave and convex portions of the over-coating layer 108 respectively. In addition, the light emitted from the light emitting device OLED passes through the substrate 100 while it is reflected and re-reflected between the surfaces of the anode 110 and the cathode 200 of the light emitting device OLED due to the irregularities, and the light passes through the substrate 100 to improve the effect of extracting light to the outside.

A bank 119 is formed to define a light emitting region BH from which light is emitted. The light emitting device OLED includes, for example, a transparent anode 110, a cathode 200 of a reflective electrode facing the anode 110, and an organic layer OS between the anode 110 and the cathode 200.

The anode 110 is divided into respective subpixels, and the remaining layers of the white-light emitting device OLED may be integrally provided in the entire display area, without being divided into respective subpixels.

As shown in FIG. 3A, in the anode 110 divided for respective subpixels (R_SP, G_SP, B_SP, W_SP), the lens is repeatedly formed on the surface thereof. As shown in FIG. 3B, on the cross-section of the over-coating layer 108 including the light emitting device OLED, the distance h between the top and bottom of the irregularities is greater than the thickness of the organic layer OS between the anode 110 and the cathode 200.

As shown in FIG. 3B, the distance h between the top and the bottom of the irregularities on the surface of the over-coating layer 108 may be greater than the distance between the anode 110 and the cathode 200 of the light emitting device OLED. Thus, for one or more concave portions, a distance from a top portion of a concave portion to a bottom portion of the concave portion h may be greater than a thickness of the organic layer OS or the distance between the anode 110 and the cathode 200.

The anode 110 includes a transparent electrode and the cathode 200 includes a reflective electrode, and thus light may be emitted toward the substrate 100.

The substrate 100 may include at least one thin film transistor (TFT) in a non-light emitting region of the subpixel and color filters 109R, 109G and 109B in the light emitting region of the subpixel.

The light generated by the light emitting device OLED may be emitted through the irregularities of the surface of the over-coating layer 108, the color filters 109R, 109G and 109B, and the substrate 100.

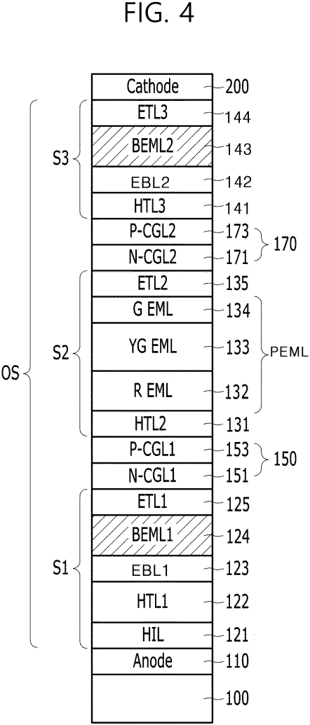

FIG. 4 is a cross-sectional view illustrating the light emitting device shown in FIG. 2 according to one embodiment, used in the first to third experimental example groups.

As shown in FIG. 4, the light emitting device used for the light emitting display device according to an embodiment of the present disclosure includes an anode 110 and a cathode 200 facing each other, first to third stacks S1, S2, S3 provided between the anode 110 and the cathode 200, and charge generation layers 150 and 170 provided between the respective stacks.

The first stack S1 is a stack emitting blue light, and includes a hole injection layer 121, a first hole transport layer HTL1 122, a first electron-blocking layer 123, a blue light emitting layer BEML1 124 containing a boron-based dopant having an emission peak of 420 nm to 480 nm, and a first electron transport layer ETL1 125 contacting the blue light emitting layer BEML1 124.

In addition, the first electron transport layer (ETL1) 125 includes the first material C1 that has excellent electron transport efficiency and is represented by Formula 1. The excellent efficiency of the material represented by Formula 1 will be described based on the following experiments. Briefly, the outermost nitrogen components present on the left side in the structure of Formula 1 cause rapid movement of electrons to the first blue light emitting layer 124 based on electron donor properties, thus leading to primary fluorescence through recombination between holes and electrons. Also, the outermost nitrogen components smoothly transfer energy to the electrons supplied from the first electron transport layer 125 to trigger delayed fluorescence by triplet-triplet annihilation (TTA) due to the coupling between triplet excitons, thereby increasing the external quantum efficiency of the first blue light emitting layer 124.

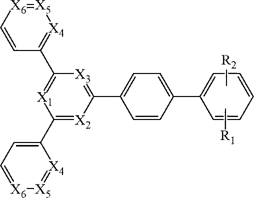

R₁ and R₂ are each independently selected from a cycloalkyl group, an aryl group, a heteroaryl group, and a carbazole group. In one embodiment, R₁ and R₂ are each independently selected from one or more phenyl rings, 3-phenyl-carbazole, and a biphenyl group. In one embodiment, X₁, X₂, and X₃ are each independently N or CH. For example, in one instance, X₁ is N and X₂ and X₃ are CH. In another instance, X₁ and X₂ are N and X₃ is CH. In another instance, X₁ and X₃ are N and X₂ is CH. In another instance, X₂ and X₃ are N and X1 is CH. In one embodiment, at least one of X₄, X₅, and X₆ must be N, and the remaining ones are CH. For example, in one instance, X₆ is N and X₄ and X₅ are CH. In another instance, X₅ is N and X₆ and X₄ are CH. In another instance, X₄ is N and X₅ and X₆ are CH.

Here, the first blue light emitting layer BEML1 of the first stack S1 has a vertical distance of 100 nm to 300 nm from the upper surface of the anode 110, and the distance is decreased by ⅒ or less compared to the surface irregularities of the over-coating layer 108 disposed thereunder.

Also, the second stack S2 includes a red-light emitting layer R EML 132 having an emission peak of 600 nm to 660 nm on the second hole transport layer (HTL2) 131, a yellow/green-light emitting layer (YG EML) 133 having an emission peak of 530 nm to 600 nm, a green-light emitting layer (G EML) 134 having an emission peak of 500 nm to 530 nm, and a second electron transport layer 135 laminated in that order.

Here, the red-light emitting layer 132, the yellow/green-light emitting layer 133, and the green-light emitting layer 134 are phosphorescent light emitting layers (PEMLs) that can share excitons with one another.

A light emitting layer that emits light having a longer wavelength than that of the phosphorescent light emitting layer PEML may be close to the anode 110.

In addition, the third stack S3 is a stack emitting blue light, and includes a third hole transport layer 141, a second electron-blocking layer 142, a second blue light emitting layer 143, and a third electron transport layer 144, similar to the first stack S1. In addition, the third stack S3 may include an electron injection layer in contact with the cathode 200. The electron injection layer is a very thin layer formed of an alkali metal, alkaline earth metal, transition metal or metal alloy, or an inorganic compound including metal fluoride such as LiF. It is also considered a component in the cathode 200 because it may be formed by adjusting the initial material in the step of forming the cathode 200.

Here, the third electron transport layer 144 may include a first material C1 having excellent efficiency represented by Formula 1 in order to realize luminous efficiency of the second blue light emitting layer 143 comparable to that of the second stack S2, which is phosphorescent, similar to that described above.

The third electron transport layer 144 is in contact with the cathode 200 or is in contact with the electron injection layer, and is thus in contact with an inorganic material such as a metal or fluoride or an inorganic compound. Accordingly, when only the single first material C1 is provided, a higher energy barrier is required for electron injection due to the high energy barrier at the interface, so the driving voltage may be increased. In order to prevent this, the third electron transport layer 144 may include, along with the first material (C1), a material including benzimidazole having almost no energy barrier with the electron injection layer and having interfacial consistency with the electron injection layer as a second material C2. In this case, when the first material C1 and the second material C2 are present in equal amounts in the third electron transport layer 144, the first material C1 can function to effectively improve efficiency and the second material C2 can effectively function to prevent the increase of the energy barrier of the electron injection layer.

Here, an electron injection layer formed of an inorganic material or an inorganic compound may be further included between the second electron transport layer and the cathode.

Meanwhile, the first and second charge generation layers 150 and 170 respectively include n-type charge generation layers 151 and 171 and p-type charge generation layers 153 and 173 that are laminated, and the n-type charge generation layers 151 and 171 generate electrons and supply the electrons to the lower stack, whereas the p-type charge generation layers 153 and 173 generate holes and supply the holes to the upper stack.

The at least two blue stacks S1 and S3 may include a first blue stack in contact with the anode 110 and a second blue stack in contact with the cathode 200. In addition, the third electron transport layer 144 of the second blue stack may further include a second material C2 of a compound including benzimidazole, compared to the first electron transport layer of the first blue stack.

The first and second blue light emitting layers BEML1 and BEML2 may include a boron-based dopant. This has an emission peak of 420 nm to 480 nm to maintain high efficiency and a lifetime suitable for the phosphorescent stack.

The blue stack further includes an electron-blocking layer 123 or 142 on a surface opposite each blue light emitting layer on which an electron transport layer is not formed, and the electron-blocking layer may be adjacent to the hole transport layer 122 or 141 disposed on the surface opposite the blue light emitting layer.

The light emitting device further includes charge generation layers 150 and 170 including a stack of an n-type charge generation layer and a p-type charge generation layer between the blue stacks S1 and S3 and the phosphorescent stack S2, the n-type charge generation layers 151 and 171 are adjacent to the electron transport layers 125 and 135 of the adjacent blue stack or the phosphorescent stack, respectively, and the p-type charge generation layers 153 and 173 are adjacent to the hole transport layers 131 and 141 of the adjacent blue stack or the phosphorescent stack.

Two out of three stacks of the light emitting device of the present disclosure shown in FIG. 4 are stacks for emitting blue light. In some cases, if a high color temperature is required in the light emitting display device, another blue stack may be further provided. That is, the light emitting device of the present disclosure is not limited to the three stacks shown in FIG. 4, and may include a plurality of stacks, including four or more stacks.

Also, in the light emitting device of the present disclosure, in the structure to which the microlens array is applied, the efficiency of emission of long-wavelength colors from the phosphorescent light emitting layer (PEML) is improved, but the blue light emission efficiency is not improved through the structure of the microlens array. In consideration thereof, the efficiency of emission of blue light from the first and second blue light emitting layers 124 and 143 is improved within the device structure by changing the material included in the electron transport layers 125 and 144 provided in the blue stacks S1 and S3.

Hereinafter, the effects of changing the material of the electron transport layers 125 and 144 in the blue stacks S1 and S3 will be described through the experiments of the first to third experimental example groups.

In the first experimental group, the first electron transport layer 125 of the first blue stack S1 includes, as a single material, ZADN of Formula 2 or a material selected from ETL1 to ETL6 below. In addition, the third electron transport layer 144 of the second blue stack S3 includes a single material of ZADN or a combination of ZADN with a material selected from ETL1 to ETL6.

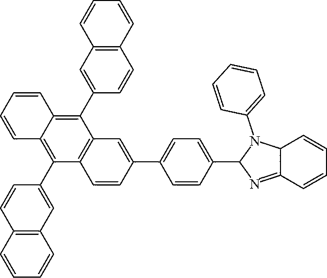 [SMILES: C1=CC2=NC(c3ccc(-c4ccc5c(-c6ccc7ccccc7c6)c6ccccc6c(-c6ccc7ccccc7c6)c5c4)cc3)N(c3ccccc3)C2C=C1]

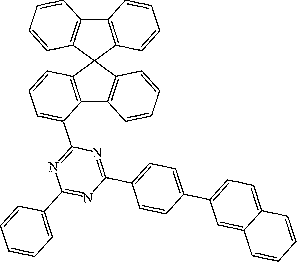 [SMILES: c1ccc(-c2nc(-c3ccc(-c4ccc5ccccc5c4)cc3)nc(-c3cccc4c3-c3ccccc3C43c4ccccc4-c4ccccc43)n2)cc1]

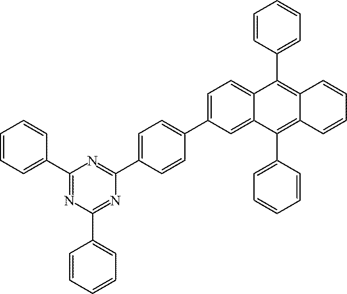 [SMILES: c1ccc(-c2nc(-c3ccccc3)nc(-c3ccc(-c4ccc5c(-c6ccccc6)c6ccccc6c(-c6ccccc6)c5c4)cc3)n2)cc1]

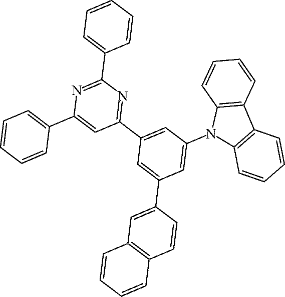 [SMILES: c1ccc(-c2cc(-c3cc(-c4ccc5ccccc5c4)cc(-n4c5ccccc5c5ccccc54)c3)nc(-c3ccccc3)n2)cc1]

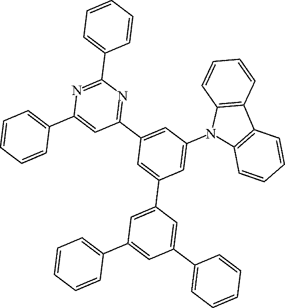 [SMILES: c1ccc(-c2cc(-c3ccccc3)cc(-c3cc(-c4cc(-c5ccccc5)nc(-c5ccccc5)n4)cc(-n4c5ccccc5c5ccccc54)c3)c2)cc1]

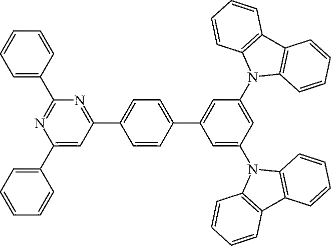 [SMILES: c1ccc(-c2cc(-c3ccc(-c4cc(-n5c6ccccc6c6ccccc65)cc(-n5c6ccccc6c6ccccc65)c4)cc3)nc(-c3ccccc3)n2)cc1]

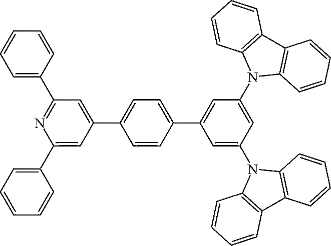 [SMILES: c1ccc(-c2cc(-c3ccc(-c4cc(-n5c6ccccc6c6ccccc65)cc(-n5c6ccccc6c6ccccc65)c4)cc3)cc(-c3ccccc3)n2)cc1]

Specifically, the first experimental example group (Ex1-1 to Ex1-25) is formed in the following process.

That is, first, a hole injection layer 121 is formed to a thickness of 5 nm using MgF₂ on an anode 110 made of ITO on a substrate.

Then, a first hole transport layer 122 is formed to a thickness of 100 nm using DNTPD of Formula 3.

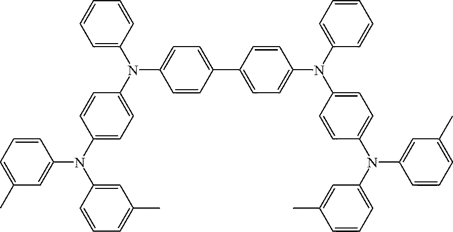 [SMILES: Cc1cccc(N(c2ccc(N(c3ccccc3)c3ccc(-c4ccc(N(c5ccccc5)c5ccc(N(c6cccc(C)c6)c6cccc(C)c6)cc5)cc4)cc3)cc2)c2cccc(C)c2)c1]

Then, a first electron-blocking layer 123 is formed to a thickness of 5 nm using TCTA of Formula 4.

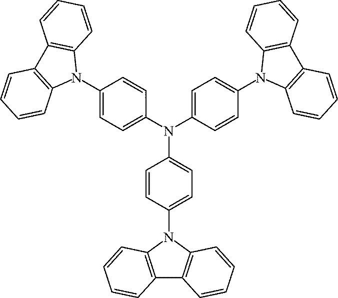 [SMILES: c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

Then, a first blue light emitting layer 124 is formed to a thickness of 20 nm by doping MADN of Formula 5 as a host at 5 wt% with DABNA-1 of Formula 6.

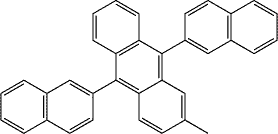 [SMILES: Cc1ccc2c(-c3ccc4ccccc4c3)c3ccccc3c(-c3ccc4ccccc4c3)c2c1]

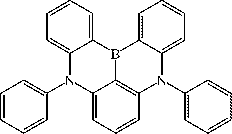 [SMILES: c1ccc(N2c3ccccc3B3c4ccccc4N(c4ccccc4)c4cccc2c43)cc1]

Then, a first electron transport layer 125 is formed to a thickness of 15 nm by changing the material to ZADN or ETL-01 to ETL-06.

Then, an n-type charge generating layer 151 is formed to a thickness of 15 nm by doping Bphen of Formula 7 as a host with Li at 2 wt%.

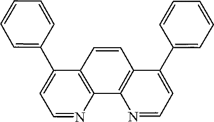 [SMILES: c1ccc(-c2ccnc3c2ccc2c(-c4ccccc4)ccnc23)cc1]

Then, a p-type charge generation layer 153 is formed to a thickness of 7 nm by doping DNTPD as a host with a p-type dopant at 20 wt%.

Then, a second hole transport layer 131 is formed to a thickness of 20 nm using BPBPA of Formula 8.

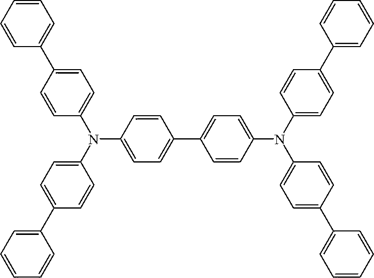 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

Then, a red-light emitting layer 132 is formed to a thickness of 10 nm by doping BPBPA and TPBi of Formula 9 at a ratio of 1:1 as a co-host at 5 wt% with Ir(piq)₂acac of Formula 10.

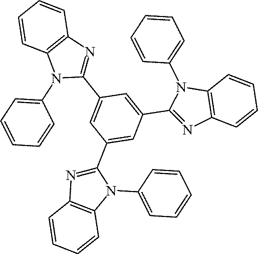 [SMILES: c1ccc(-n2c(-c3cc(-c4nc5ccccc5n4-c4ccccc4)cc(-c4nc5ccccc5n4-c4ccccc4)c3)nc3ccccc32)cc1]

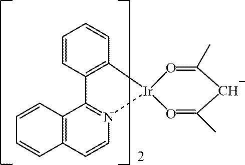

Next, a yellow/green-light emitting layer 133 is formed to a thickness of 10 nm by doping CBP and TPBi of Formula 11 at a ratio of 1:1 as a co-host at 15 wt% with PO-01 of Formula 12.

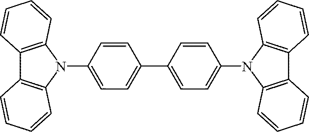 [SMILES: c1ccc2c(c1)c1ccccc1n2-c1ccc(-c2ccc(-n3c4ccccc4c4ccccc43)cc2)cc1]

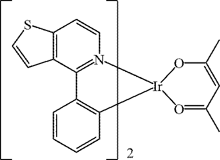

Next, a green-light emitting layer 134 is formed to a thickness of 20 nm by doping CBP and TPBi at a ratio of 1:1 as a co-host at 15 wt% with Ir(ppy)₃ of Formula 13.

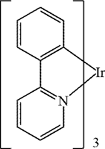

Next, a second electron transport layer 135 is formed to a thickness of 20 nm using TPBi.

Next, an n-type charge generation layer 171 is formed to a thickness of 20 nm by doping Bphen as a host at 3 wt% with Li.

Then, a p-type charge generation layer 173 is formed to a thickness of 10 nm by doping DNTPD as a host at 20 wt% with a p-type dopant.

Then, a third hole transport layer 141 is formed to a thickness of 100 nm using DNTPD.

Then, a second electron-blocking layer 142 is formed to a thickness of 5 nm using TCTA.

Then, a second blue light emitting layer 143 is formed to a thickness of 20 nm by doping MADN as a host at 5 wt% with DABNA-1.

Then, a third electron transport layer ETL3 144 of a first experimental example group is formed using ZADN as a first component (BETL2-1) or any one of ETL-01 to ETL-06 as a second component (BETL2-2), if present. When the first and second components (BETL2-1, BETL2-2) were used in combination, the ratio therebetween was set to 5:5.

Next, LiF is formed to a thickness of 1.5 nm to form an electron injection layer.

Next, Al is deposited to a thickness of 100 nm to form a cathode 200.

The experimental results for the first experimental group (Ex1-1 to Ex1-25) will be described with reference to Table 1 below.

TABLE 1

| | ETL1 (125) | ETL3 (144) (BETL2-1: BETL2-2) | Voltage @10 mA/cm2 (V) | R Efficiency @10 mA/cm² (cd/A) | G Efficiency @10 mA/cm² (cd/A) | B Efficiency @10 mA/cm² (cd/A) | W Efficiency @10 mA/cm² (cd/A) | CIEx | CIEy |
|---|---|---|---|---|---|---|---|---|---|
| Ex1 -1 | ZADN | ZADN | 12.12 | 10.4 | 25.48 | 4.79 | 71.6 | 0.300 | 0.311 |
| Ex1 -2 | ZADN | ZADN:ET L-01 | 12.10 | 10.3 | 25.17 | 4.66 | 70.8 | 0.302 | 0.314 |
| Ex1 -3 | ZADN | ZADN:ET L-02 | 12.01 | 10.4 | 25.29 | 4.69 | 71.1 | 0.302 | 0.314 |
| Ex1 -4 | ZADN | ZADN:ET L-03 | 12.11 | 10.4 | 25.34 | 4.73 | 71.2 | 0.301 | 0.313 |
| Ex1 -5 | ZADN | ZADN:ET L-04 | 12.20 | 10.5 | 25.20 | 4.6 | 70.9 | 0.305 | 0.317 |
| Ex1 -6 | ZADN | ZADN:ET L-05 | 12.04 | 10.3 | 25.21 | 4.7 | 70.9 | 0.302 | 0.314 |
| Ex1 -7 | ZADN | ZADN:ET L-06 | 12.11 | 10.4 | 25.42 | 4.8 | 71.5 | 0.299 | 0.310 |
| Ex1 -8 | ETL -01 | ZADN | 12.13 | 10.4 | 25.51 | 4.8 | 71.7 | 0.301 | 0.313 |
| Ex1 -9 | ETL -02 | ZADN | 12.05 | 10.5 | 25.59 | 4.8 | 72.0 | 0.300 | 0.312 |
| Ex1 -10 | ETL -03 | ZADN | 12.15 | 10.4 | 25.36 | 4.7 | 71.3 | 0.301 | 0.313 |
| Ex1 -11 | ETL -04 | ZADN | 12.03 | 10.3 | 25.54 | 4.8 | 71.7 | 0.299 | 0.311 |
| Ex1 -12 | ETL -05 | ZADN | 12.16 | 10.3 | 25.31 | 4.7 | 71.0 | 0.301 | 0.314 |
| Ex1 -13 | ETL -06 | ZADN | 12.19 | 10.5 | 25.36 | 4.7 | 71.5 | 0.304 | 0.315 |
| Ex1 -14 | ETL -03 | ZADN:ET L-01 | 12.04 | 10.3 | 25.19 | 4.6 | 70.8 | 0.302 | 0.315 |
| Ex1 -15 | ETL -03 | ZADN:ET L-02 | 12.16 | 10.4 | 25.46 | 4.7 | 71.6 | 0.302 | 0.314 |
| Ex1 -16 | ETL -03 | ZADN:ET L-03 | 12.19 | 10.5 | 25.51 | 4.7 | 71.8 | 0.304 | 0.317 |
| Ex1 -17 | ETL -03 | ZADN:ET L-04 | 12.16 | 10.3 | 25.14 | 4.7 | 70.7 | 0.301 | 0.313 |
| Ex1 -18 | ETL -03 | ZADN:ET L-05 | 12.19 | 10.5 | 25.20 | 4.7 | 71.0 | 0.301 | 0.312 |
| Ex1 -19 | ETL -03 | ZADN:ET L-06 | 12.19 | 10.3 | 25.53 | 4.8 | 71.6 | 0.299 | 0.311 |
| Ex1 -20 | ETL -05 | ZADN:ET L-01 | 12.18 | 10.4 | 25.51 | 4.6 | 71.7 | 0.305 | 0.318 |
| Ex1 -21 | ETL -05 | ZADN:ET L-02 | 12.09 | 10.5 | 25.49 | 4.7 | 71.8 | 0.304 | 0.317 |
| Ex1 -22 | ETL -05 | ZADN:ET L-03 | 12.13 | 10.5 | 25.48 | 4.6 | 71.7 | 0.305 | 0.318 |
| Ex1 -23 | ETL -05 | ZADN:ET L-04 | 12.06 | 10.4 | 25.44 | 4.7 | 71.5 | 0.303 | 0.315 |
| Ex1 -24 | ETL -05 | ZADN:ET L-05 | 12.01 | 10.4 | 25.49 | 4.6 | 71.6 | 0.305 | 0.319 |
| Ex1 -25 | ETL -05 | ZADN:ET L-06 | 12.09 | 10.4 | 25.52 | 4.6 | 71.7 | 0.304 | 0.317 |

In the first experimental examples (Ex1-1 to Ex-25) in Table 1, the driving voltage, red light emission efficiency, green light emission efficiency, blue light emission efficiency, and white light emission efficiency from all three stacks at a current density of 10 mA/cm² were evaluated through experimentation. The values of the white color coordinates in the white subpixel, after passing through the microlens array on the final light-emission region, were evaluated. As can be seen from Table 1, electron transport layers ETL1 and ETL2 of the first and second blue stacks in the first experimental example group (Ex1-1 to Ex-25) include ZADN alone or a combination thereof. In particular, it can be seen that all CIEx values of the evaluated white color coordinates are higher than 0.300 and all CIEy values thereof are 0.310 or more, which is considered to be due to the decrease in blue light emission efficiency.

Hereinafter, the light emitting display device of the present disclosure includes the first material C1 of Formula 1 in at least the electron transport layer of the blue stack in order to improve blue efficiency.

Examples of the first material C1 of Formula 1 include ETM-01 to ETM-60 shown below. In addition, at least one of the first material C1 of Formula 1 may be present in the first electron transport layer 125 and/or the third electron transport layer 144 of the blue stacks S1 and S3.

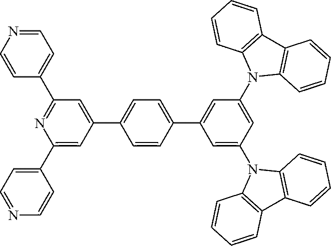 [SMILES: c1ccc2c(c1)c1ccccc1n2-c1cc(-c2ccc(-c3cc(-c4ccncc4)nc(-c4ccncc4)c3)cc2)cc(-n2c3ccccc3c3ccccc32)c1]

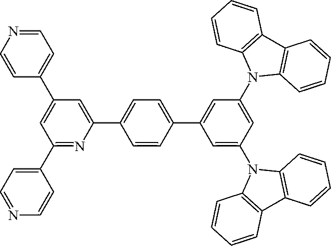 [SMILES: c1ccc2c(c1)c1ccccc1n2-c1cc(-c2ccc(-c3cc(-c4ccncc4)cc(-c4ccncc4)n3)cc2)cc(-n2c3ccccc3c3ccccc32)c1]

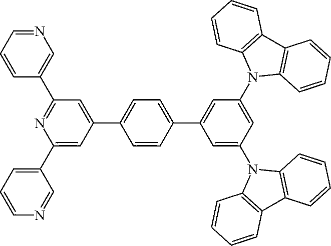 [SMILES: c1cncc(-c2cc(-c3ccc(-c4cc(-n5c6ccccc6c6ccccc65)cc(-n5c6ccccc6c6ccccc65)c4)cc3)cc(-c3cccnc3)n2)c1]

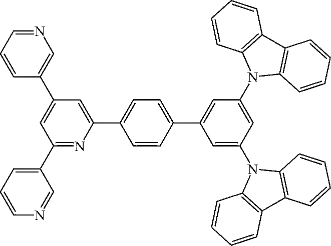 [SMILES: c1cncc(-c2cc(-c3ccc(-c4cc(-n5c6ccccc6c6ccccc65)cc(-n5c6ccccc6c6ccccc65)c4)cc3)nc(-c3cccnc3)c2)c1]

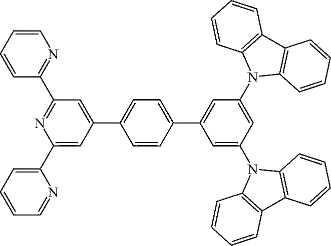 [SMILES: c1ccc(-c2cc(-c3ccc(-c4cc(-n5c6ccccc6c6ccccc65)cc(-n5c6ccccc6c6ccccc65)c4)cc3)cc(-c3ccccn3)n2)nc1]

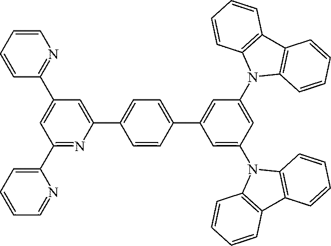 [SMILES: c1ccc(-c2cc(-c3ccc(-c4cc(-n5c6ccccc6c6ccccc65)cc(-n5c6ccccc6c6ccccc65)c4)cc3)nc(-c3ccccn3)c2)nc1]

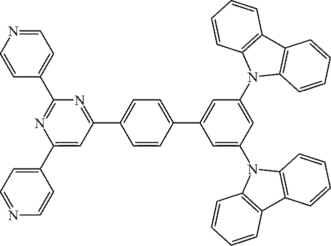 [SMILES: c1ccc2c(c1)c1ccccc1n2-c1cc(-c2ccc(-c3cc(-c4ccncc4)nc(-c4ccncc4)n3)cc2)cc(-n2c3ccccc3c3ccccc32)c1]

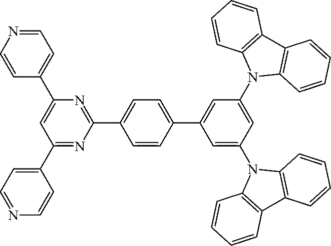 [SMILES: c1ccc2c(c1)c1ccccc1n2-c1cc(-c2ccc(-c3nc(-c4ccncc4)cc(-c4ccncc4)n3)cc2)cc(-n2c3ccccc3c3ccccc32)c1]

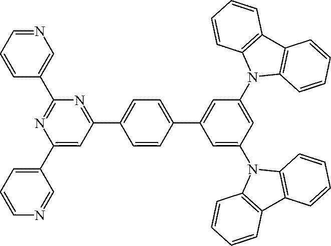 [SMILES: c1cncc(-c2cc(-c3ccc(-c4cc(-n5c6ccccc6c6ccccc65)cc(-n5c6ccccc6c6ccccc65)c4)cc3)nc(-c3cccnc3)n2)c1]

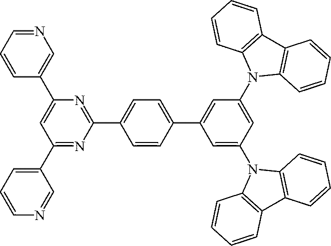 [SMILES: c1cncc(-c2cc(-c3cccnc3)nc(-c3ccc(-c4cc(-n5c6ccccc6c6ccccc65)cc(-n5c6ccccc6c6ccccc65)c4)cc3)n2)c1]

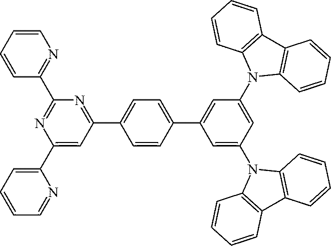 [SMILES: c1ccc(-c2cc(-c3ccc(-c4cc(-n5c6ccccc6c6ccccc65)cc(-n5c6ccccc6c6ccccc65)c4)cc3)nc(-c3ccccn3)n2)nc1]

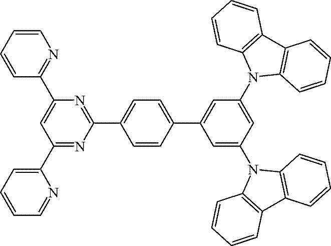 [SMILES: c1ccc(-c2cc(-c3ccccn3)nc(-c3ccc(-c4cc(-n5c6ccccc6c6ccccc65)cc(-n5c6ccccc6c6ccccc65)c4)cc3)n2)nc1]

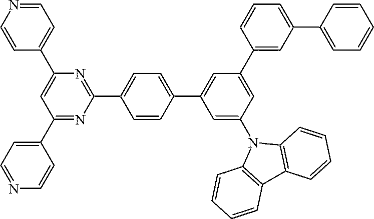 [SMILES: c1ccc(-c2cccc(-c3cc(-c4ccc(-c5nc(-c6ccncc6)cc(-c6ccncc6)n5)cc4)cc(-n4c5ccccc5c5ccccc54)c3)c2)cc1]

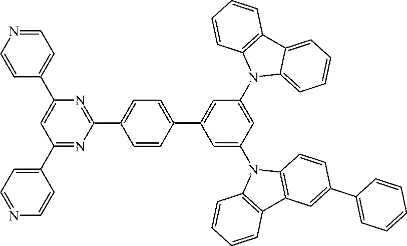 [SMILES: c1ccc(-c2ccc3c(c2)c2ccccc2n3-c2cc(-c3ccc(-c4nc(-c5ccncc5)cc(-c5ccncc5)n4)cc3)cc(-n3c4ccccc4c4ccccc43)c2)cc1]

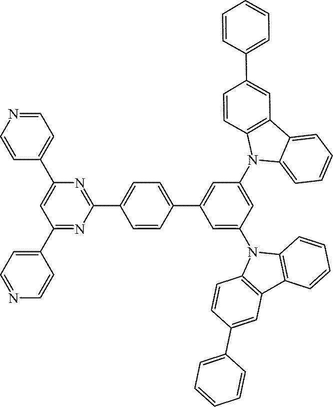 [SMILES: c1ccc(-c2ccc3c(c2)c2ccccc2n3-c2cc(-c3ccc(-c4nc(-c5ccncc5)cc(-c5ccncc5)n4)cc3)cc(-n3c4ccccc4c4cc(-c5ccccc5)ccc43)c2)cc1]

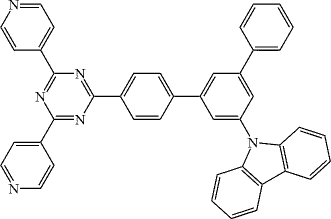 [SMILES: c1ccc(-c2cc(-c3ccc(-c4nc(-c5ccncc5)nc(-c5ccncc5)n4)cc3)cc(-n3c4ccccc4c4ccccc43)c2)cc1]

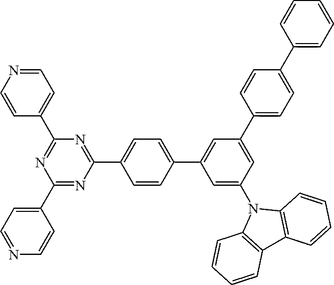 [SMILES: c1ccc(-c2ccc(-c3cc(-c4ccc(-c5nc(-c6ccncc6)nc(-c6ccncc6)n5)cc4)cc(-n4c5ccccc5c5ccccc54)c3)cc2)cc1]

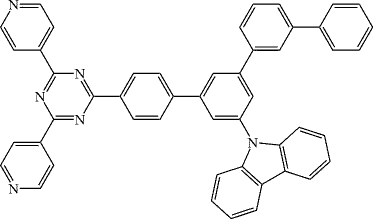 [SMILES: c1ccc(-c2cccc(-c3cc(-c4ccc(-c5nc(-c6ccncc6)nc(-c6ccncc6)n5)cc4)cc(-n4c5ccccc5c5ccccc54)c3)c2)cc1]

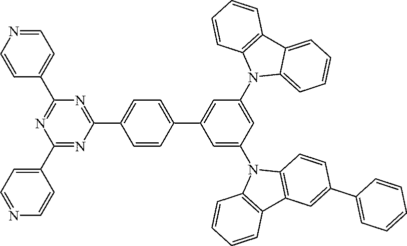 [SMILES: c1ccc(-c2ccc3c(c2)c2ccccc2n3-c2cc(-c3ccc(-c4nc(-c5ccncc5)nc(-c5ccncc5)n4)cc3)cc(-n3c4ccccc4c4ccccc43)c2)cc1]

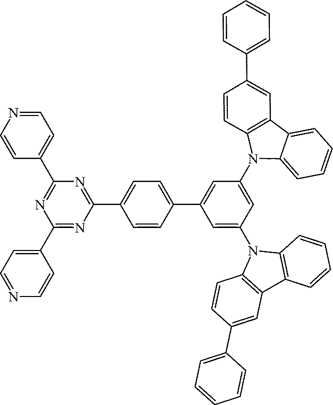 [SMILES: c1ccc(-c2ccc3c(c2)c2ccccc2n3-c2cc(-c3ccc(-c4nc(-c5ccncc5)nc(-c5ccncc5)n4)cc3)cc(-n3c4ccccc4c4cc(-c5ccccc5)ccc43)c2)cc1]

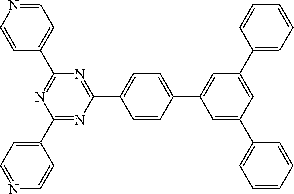 [SMILES: c1ccc(-c2cc(-c3ccccc3)cc(-c3ccc(-c4nc(-c5ccncc5)nc(-c5ccncc5)n4)cc3)c2)cc1]

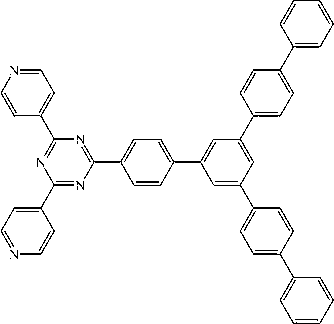 [SMILES: c1ccc(-c2ccc(-c3cc(-c4ccc(-c5ccccc5)cc4)cc(-c4ccc(-c5nc(-c6ccncc6)nc(-c6ccncc6)n5)cc4)c3)cc2)cc1]

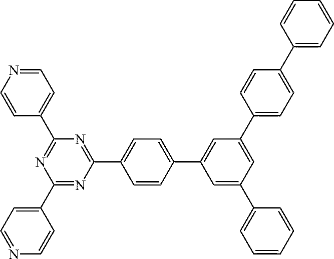 [SMILES: c1ccc(-c2ccc(-c3cc(-c4ccccc4)cc(-c4ccc(-c5nc(-c6ccncc6)nc(-c6ccncc6)n5)cc4)c3)cc2)cc1]

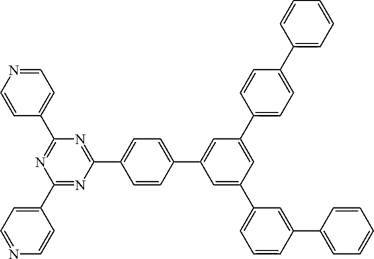 [SMILES: c1ccc(-c2ccc(-c3cc(-c4ccc(-c5nc(-c6ccncc6)nc(-c6ccncc6)n5)cc4)cc(-c4cccc(-c5ccccc5)c4)c3)cc2)cc1]

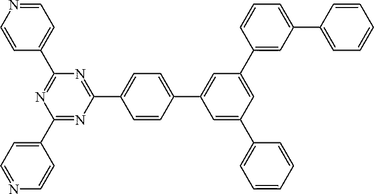 [SMILES: c1ccc(-c2cccc(-c3cc(-c4ccccc4)cc(-c4ccc(-c5nc(-c6ccncc6)nc(-c6ccncc6)n5)cc4)c3)c2)cc1]

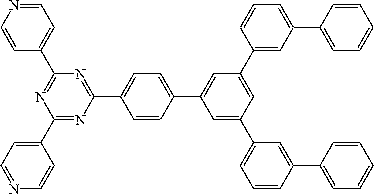 [SMILES: c1ccc(-c2cccc(-c3cc(-c4ccc(-c5nc(-c6ccncc6)nc(-c6ccncc6)n5)cc4)cc(-c4cccc(-c5ccccc5)c4)c3)c2)cc1]

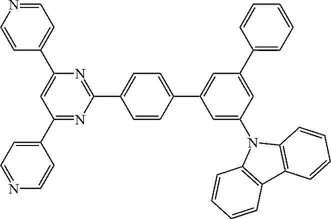 [SMILES: c1ccc(-c2cc(-c3ccc(-c4nc(-c5ccncc5)cc(-c5ccncc5)n4)cc3)cc(-n3c4ccccc4c4ccccc43)c2)cc1]

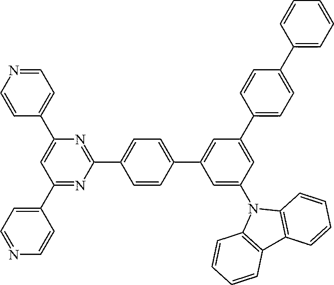 [SMILES: c1ccc(-c2ccc(-c3cc(-c4ccc(-c5nc(-c6ccncc6)cc(-c6ccncc6)n5)cc4)cc(-n4c5ccccc5c5ccccc54)c3)cc2)cc1]

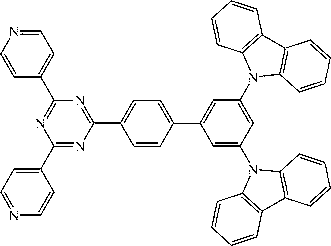 [SMILES: c1ccc2c(c1)c1ccccc1n2-c1cc(-c2ccc(-c3nc(-c4ccncc4)nc(-c4ccncc4)n3)cc2)cc(-n2c3ccccc3c3ccccc32)c1]

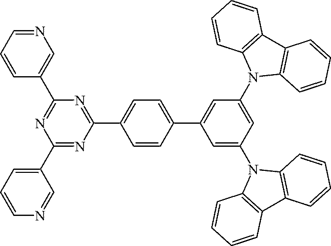 [SMILES: c1cncc(-c2nc(-c3ccc(-c4cc(-n5c6ccccc6c6ccccc65)cc(-n5c6ccccc6c6ccccc65)c4)cc3)nc(-c3cccnc3)n2)c1]

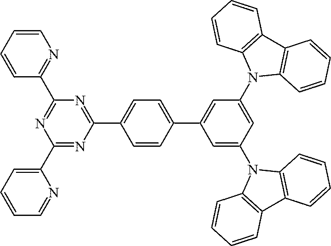 [SMILES: c1ccc(-c2nc(-c3ccc(-c4cc(-n5c6ccccc6c6ccccc65)cc(-n5c6ccccc6c6ccccc65)c4)cc3)nc(-c3ccccn3)n2)nc1]

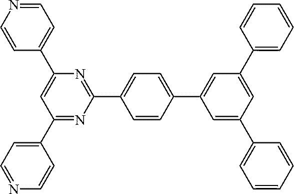 [SMILES: c1ccc(-c2cc(-c3ccccc3)cc(-c3ccc(-c4nc(-c5ccncc5)cc(-c5ccncc5)n4)cc3)c2)cc1]

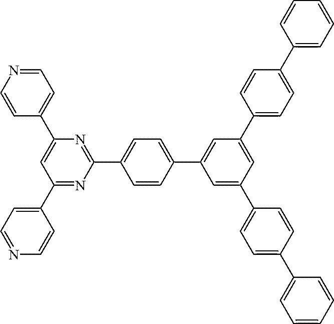 [SMILES: c1ccc(-c2ccc(-c3cc(-c4ccc(-c5ccccc5)cc4)cc(-c4ccc(-c5nc(-c6ccncc6)cc(-c6ccncc6)n5)cc4)c3)cc2)cc1]

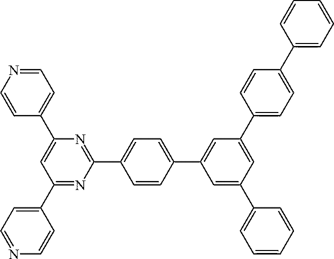 [SMILES: c1ccc(-c2ccc(-c3cc(-c4ccccc4)cc(-c4ccc(-c5nc(-c6ccncc6)cc(-c6ccncc6)n5)cc4)c3)cc2)cc1]

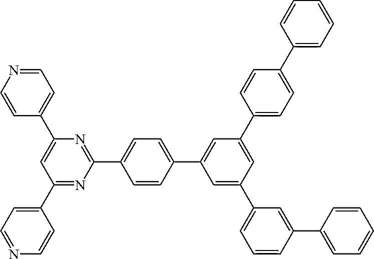 [SMILES: c1ccc(-c2ccc(-c3cc(-c4ccc(-c5nc(-c6ccncc6)cc(-c6ccncc6)n5)cc4)cc(-c4cccc(-c5ccccc5)c4)c3)cc2)cc1]

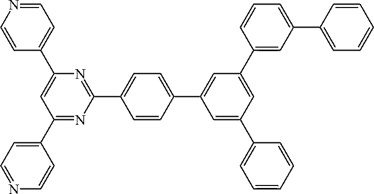 [SMILES: c1ccc(-c2cccc(-c3cc(-c4ccccc4)cc(-c4ccc(-c5nc(-c6ccncc6)cc(-c6ccncc6)n5)cc4)c3)c2)cc1]

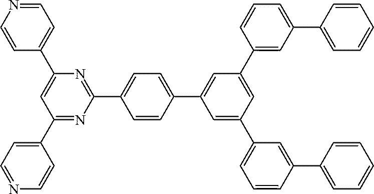 [SMILES: c1ccc(-c2cccc(-c3cc(-c4ccc(-c5nc(-c6ccncc6)cc(-c6ccncc6)n5)cc4)cc(-c4cccc(-c5ccccc5)c4)c3)c2)cc1]

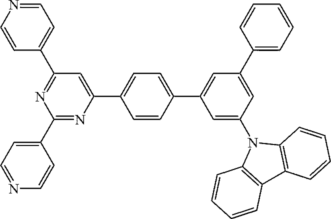 [SMILES: c1ccc(-c2cc(-c3ccc(-c4cc(-c5ccncc5)nc(-c5ccncc5)n4)cc3)cc(-n3c4ccccc4c4ccccc43)c2)cc1]

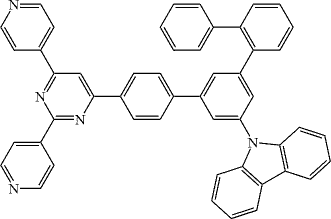 [SMILES: c1ccc(-c2ccccc2-c2cc(-c3ccc(-c4cc(-c5ccncc5)nc(-c5ccncc5)n4)cc3)cc(-n3c4ccccc4c4ccccc43)c2)cc1]

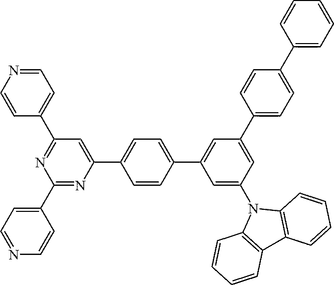 [SMILES: c1ccc(-c2ccc(-c3cc(-c4ccc(-c5cc(-c6ccncc6)nc(-c6ccncc6)n5)cc4)cc(-n4c5ccccc5c5ccccc54)c3)cc2)cc1]

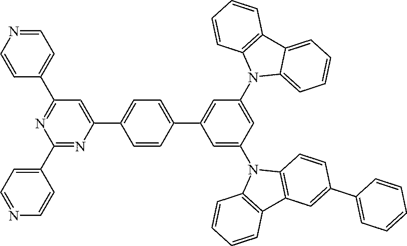 [SMILES: c1ccc(-c2ccc3c(c2)c2ccccc2n3-c2cc(-c3ccc(-c4cc(-c5ccncc5)nc(-c5ccncc5)n4)cc3)cc(-n3c4ccccc4c4ccccc43)c2)cc1]

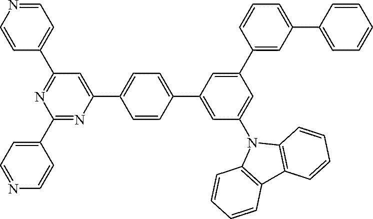 [SMILES: c1ccc(-c2cccc(-c3cc(-c4ccc(-c5cc(-c6ccncc6)nc(-c6ccncc6)n5)cc4)cc(-n4c5ccccc5c5ccccc54)c3)c2)cc1]

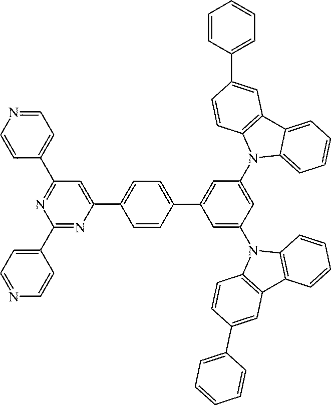 [SMILES: c1ccc(-c2ccc3c(c2)c2ccccc2n3-c2cc(-c3ccc(-c4cc(-c5ccncc5)nc(-c5ccncc5)n4)cc3)cc(-n3c4ccccc4c4cc(-c5ccccc5)ccc43)c2)cc1]

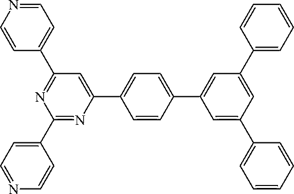 [SMILES: c1ccc(-c2cc(-c3ccccc3)cc(-c3ccc(-c4cc(-c5ccncc5)nc(-c5ccncc5)n4)cc3)c2)cc1]

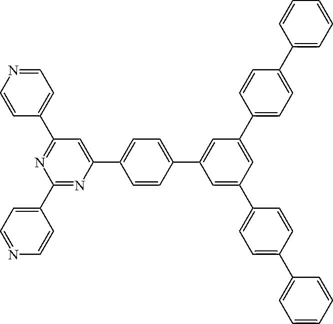 [SMILES: c1ccc(-c2ccc(-c3cc(-c4ccc(-c5ccccc5)cc4)cc(-c4ccc(-c5cc(-c6ccncc6)nc(-c6ccncc6)n5)cc4)c3)cc2)cc1]

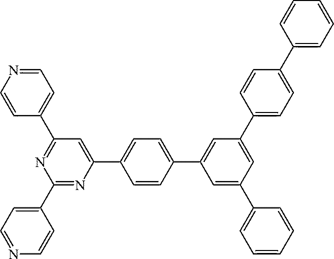 [SMILES: c1ccc(-c2ccc(-c3cc(-c4ccccc4)cc(-c4ccc(-c5cc(-c6ccncc6)nc(-c6ccncc6)n5)cc4)c3)cc2)cc1]

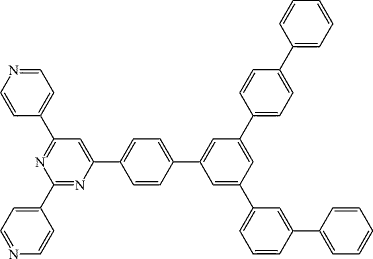 [SMILES: c1ccc(-c2ccc(-c3cc(-c4ccc(-c5cc(-c6ccncc6)nc(-c6ccncc6)n5)cc4)cc(-c4cccc(-c5ccccc5)c4)c3)cc2)cc1]

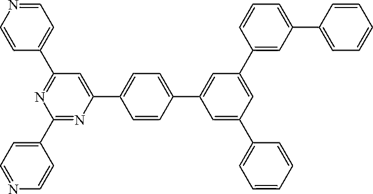 [SMILES: c1ccc(-c2cccc(-c3cc(-c4ccccc4)cc(-c4ccc(-c5cc(-c6ccncc6)nc(-c6ccncc6)n5)cc4)c3)c2)cc1]

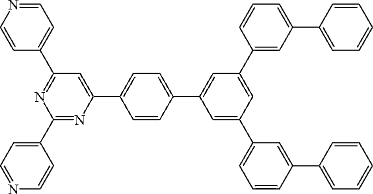 [SMILES: c1ccc(-c2cccc(-c3cc(-c4ccc(-c5cc(-c6ccncc6)nc(-c6ccncc6)n5)cc4)cc(-c4cccc(-c5ccccc5)c4)c3)c2)cc1]

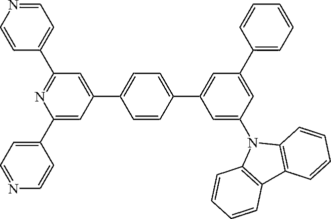 [SMILES: c1ccc(-c2cc(-c3ccc(-c4cc(-c5ccncc5)nc(-c5ccncc5)c4)cc3)cc(-n3c4ccccc4c4ccccc43)c2)cc1]

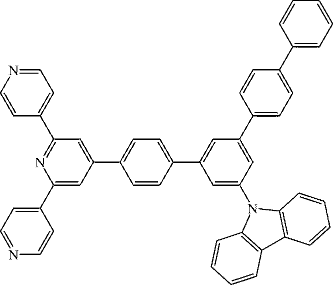 [SMILES: c1ccc(-c2ccc(-c3cc(-c4ccc(-c5cc(-c6ccncc6)nc(-c6ccncc6)c5)cc4)cc(-n4c5ccccc5c5ccccc54)c3)cc2)cc1]

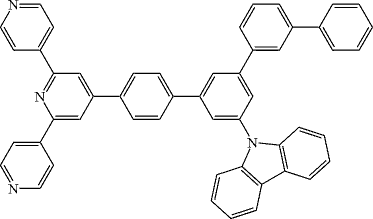 [SMILES: c1ccc(-c2cccc(-c3cc(-c4ccc(-c5cc(-c6ccncc6)nc(-c6ccncc6)c5)cc4)cc(-n4c5ccccc5c5ccccc54)c3)c2)cc1]

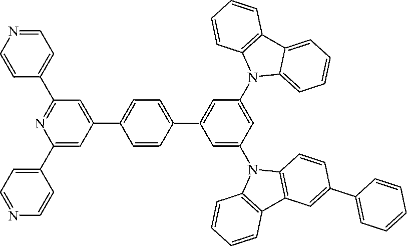 [SMILES: c1ccc(-c2ccc3c(c2)c2ccccc2n3-c2cc(-c3ccc(-c4cc(-c5ccncc5)nc(-c5ccncc5)c4)cc3)cc(-n3c4ccccc4c4ccccc43)c2)cc1]

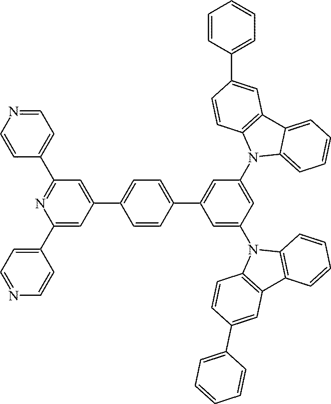 [SMILES: c1ccc(-c2ccc3c(c2)c2ccccc2n3-c2cc(-c3ccc(-c4cc(-c5ccncc5)nc(-c5ccncc5)c4)cc3)cc(-n3c4ccccc4c4cc(-c5ccccc5)ccc43)c2)cc1]

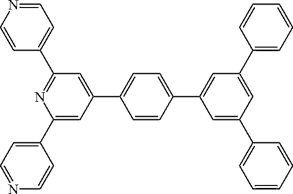 [SMILES: c1ccc(-c2cc(-c3ccccc3)cc(-c3ccc(-c4cc(-c5ccncc5)nc(-c5ccncc5)c4)cc3)c2)cc1]

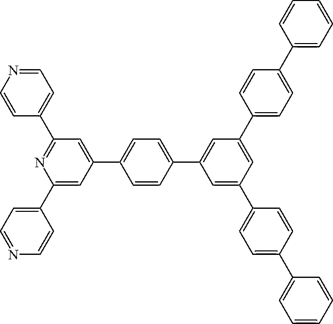 [SMILES: c1ccc(-c2ccc(-c3cc(-c4ccc(-c5ccccc5)cc4)cc(-c4ccc(-c5cc(-c6ccncc6)nc(-c6ccncc6)c5)cc4)c3)cc2)cc1]

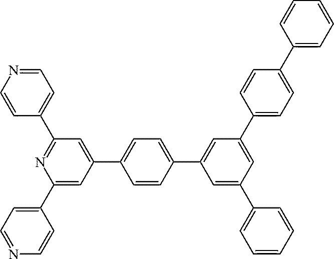 [SMILES: c1ccc(-c2ccc(-c3cc(-c4ccccc4)cc(-c4ccc(-c5cc(-c6ccncc6)nc(-c6ccncc6)c5)cc4)c3)cc2)cc1]

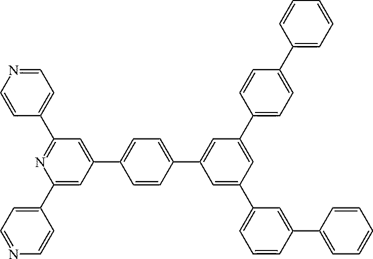 [SMILES: c1ccc(-c2ccc(-c3cc(-c4ccc(-c5cc(-c6ccncc6)nc(-c6ccncc6)c5)cc4)cc(-c4cccc(-c5ccccc5)c4)c3)cc2)cc1]

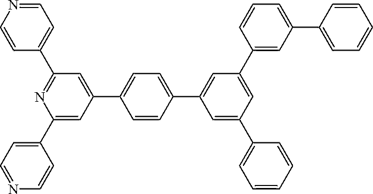 [SMILES: c1ccc(-c2cccc(-c3cc(-c4ccccc4)cc(-c4ccc(-c5cc(-c6ccncc6)nc(-c6ccncc6)c5)cc4)c3)c2)cc1]

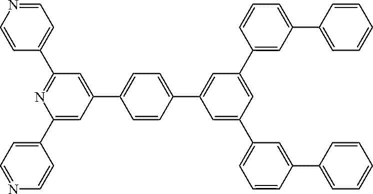 [SMILES: c1ccc(-c2cccc(-c3cc(-c4ccc(-c5cc(-c6ccncc6)nc(-c6ccncc6)c5)cc4)cc(-c4cccc(-c5ccccc5)c4)c3)c2)cc1]

As a representative material of Formula 1, ETM-01 was obtained through the following preparation method.

### (1) Synthesis of First Compound

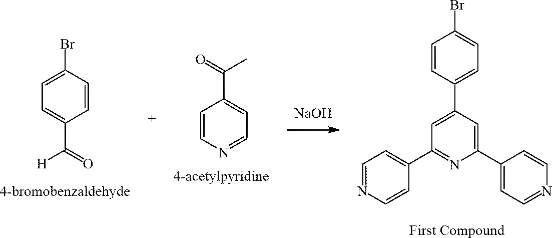

6.0 g (49.5 mmol) of 4-acetylpyridine and 9.0 g (48.6 mmol) of 4-bromobenzaldehyde were prepared and placed in a flask along with 200 ml of a 2% NaOH aqueous solution, followed by stirring at room temperature for 10 hours and observation of the color change of the reaction solution. Then, 6.0 g (49.5 mmol) of 4-acetylpyridine was added thereto, and an NaOH concentration was set to 20%, followed by stirring at 80° C. for 8 hours. The product was dehydrated without purification and stirred under reflux conditions in a solution containing more than 36.0 g of ammonium acetate in 500 mL of ethanol for 5 hours. Then, the resulting reaction solution was recrystallized from ethanol to obtain a first compound (11 g, 60%).

### (2) Synthesis of Second Compound

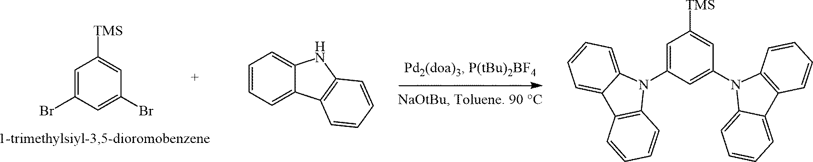

8.1 g (48.75 mmol) of Carbazole, 6.0 g (19.5 mmol) of 1-trimethylsilyl-3,5-dibromobenzene, 0.89 g (1.0 mmol) of tris(dibenzylideneacetone)dipalladium(0) (Pd₂(dba)₃), 0.59 g (2.0 mmol) of tri-tert-butylphosphine tetrafluoroborate, and 4.7 g (48.8 mmol) of NaOtBu were added to dry toluene (200 mL), followed by stirring at 90° C. for 10 hours. Upon completion of the reaction, the NaOtBu residue was filtered and then extracted with ethyl acetate. The resulting product was dehydrated with MgSO₄ and purified through column chromatography (hexane:EA = 20:1) to obtain a second compound (6.1 g, 65%).

### (3) Synthesis of Third Compound

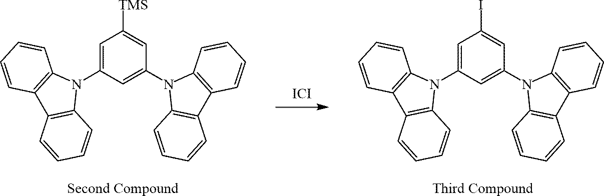

The second compound (4.0 g, 8.3 mmol) was dissolved in CCl₄ (70 mL), and an iodine monochloride solution (1.0 M in methylene chloride, 8.3 mL, 8.3 mmol) was added dropwise thereto at 0° C., followed by stirring for 1 hour. Then, the reaction solution was added to a 5 wt% aqueous solution of sodium thiosulfate (Na₂S₂O₃), followed by vigorous stirring until the reaction solution became transparent. The reaction solution was extracted with ethylene acetate, dehydrated with MsSO₄, and purified through column chromatography (hexane:EA = 20:1) to obtain a third compound (3.6 g, 80%).

### (4) Synthesis of Fourth Compound

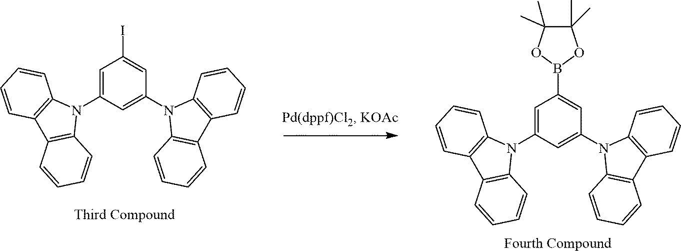

The third compound (3 g, 5.6 mmol), 4,4,4”,4′,5,5,5′,5′-octamethyl-2,2′-bi(1,3,2,-dioxaborolane) (2.1 g, 8.4 mmol), [1,1′bis(diphenylphosphino)ferrocene]dichloropalladium (II) (Pd(dppf)Cl₂) (0.21 g, 0.28 mmol), and potassium acetate (KOAc) (1.65 g, 16.8 mmol) were mixed under an inert atmosphere formed by blowing of nitrogen, and then 1,4-dioxane (30 mL) was added thereto, followed by stirring at 130° C. for 12 hours. After completion of the reaction, the mixture was extracted with ethyl acetate, dehydrated with MgSO₄, and purified through column chromatography (hexane:EA = 7:1 (v/v)) to obtain a fourth compound (2.3 g, 75%).

### (5) Synthesis of ETM-01

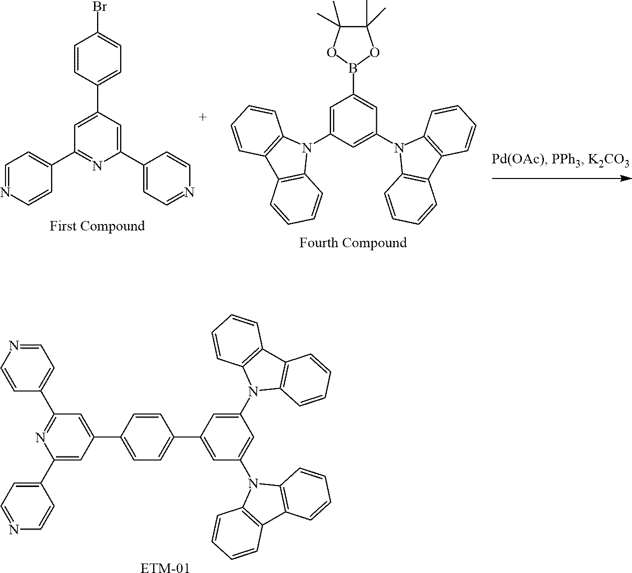

The first compound (1.8 g, 4.6 mmol), the fourth compound (2.3 g, 4.3 mmol), palladium(II) acetate (Pd(Oac)₂) (0.048 g, 0.2 mmol), triphenylphosphine (0.28 g, 1.0 mmol), and potassium carbonate (K₂CO₃) (3.0 g, 21.5 mmol) were charged in a round-bottom flask, an inert atmosphere was formed, and degassed THF/H₂O (35 mL/5 mL) was added thereto, followed by stirring at 70° C. for 10 hours. After completion of the reaction, the reaction solution was extracted with dichloromethane, dehydrated with MgSO₄, and purified through column chromatography (hexane:EA = 7:1 (v/v)) to obtain ETM-01 (2.6 g, 85%).

Meanwhile, the above synthesis method is used to synthesize the representative material ETM-01 of Formula 1, and ETM-02 to ETM-60, the materials of other Formulas, may be obtained by changing the number of nitrogen substituents during synthesis of the first compound in (1) or by changing the number of synthesized carbazoles or phenyl groups during synthesis of the second compound in (2).

The following second experimental group (Ex2-1 to Ex2-45) uses the structure of FIG. 4, but differs from the first experimental group (Ex1-1 to Ex1-25) in terms of the first electron transport layer 125 and the third electron transport layer 144.

That is, in Ex2-1 to Ex2-9 of the second experimental group (Ex2-1 to Ex2-45), the first electron transport layer 125 includes ZADN alone, and the third electron transport layer 144 includes ZADN as a first component BETL2-1 and any one of ETM-01 to ETM-09 as a second component (BETL2-2). Also, in Ex2-10 to Ex2-45, the first electron transport layer 125 includes, as a single material, any one of ETM-05, ETM-06, ETM-07, or ETM-08, but the third electron transport layer 144 includes a double material of ZADN as a first component BETL2-1 and any one of ETM-01 to ETM-09 changed as a second component BETL2-2. The first component BETL2-1 and the second component BETL2-2 of the third electron transport layer 144 are present at a ratio of 1:1.

Also, the second experimental example group (Ex2-1 to Ex2-45) including at least one of the first and third electron transport layers including the material of Formula 1 shows the results in Table 2.

TABLE 2

| | ETL 1 (125) | ETL3(144)(BETL2-1:BETL2-2) | Voltage @10 mA/cm2(V) | R Efficiency @10 mA/ cm2 (cd/A) | G Efficiency @10 mA/ cm2 (cd/A) | B Efficiency @10 mA/c m2 (cd/A) | W Efficiency @10 mA/c m2 (cd/A) | CIEx | CIEy |
|---|---|---|---|---|---|---|---|---|---|
| Ex2 -1 | ZAD N | ZADN:ETM-01 | 12.07 | 10.3 | 25.61 | 4.87 | 71.9 | 0.298 | 0.309 |
| Ex2 -2 | ZAD N | ZADN:ETM-02 | 12.19 | 10.4 | 25.46 | 4.90 | 71.7 | 0.297 | 0.307 |
| Ex2 -3 | ZAD N | ZADN:ETM-03 | 12.12 | 10.4 | 25.69 | 4.94 | 72.2 | 0.296 | 0.307 |
| Ex2 -4 | ZAD N | ZADN:ETM-04 | 12.18 | 10.4 | 25.53 | 4.87 | 71.9 | 0.298 | 0.309 |
| Ex2 -5 | ZAD N | ZADN:ETM-05 | 12.06 | 10.6 | 25.93 | 4.9 | 73.0 | 0.298 | 0.309 |
| Ex2 -6 | ZAD N | ZADN:ETM-06 | 12.11 | 10.3 | 25.47 | 4.9 | 71.6 | 0.297 | 0.307 |
| Ex2 -7 | ZAD N | ZADN:ETM-07 | 12.16 | 10.5 | 25.77 | 4.9 | 72.6 | 0.298 | 0.307 |
| Ex2 -8 | ZAD N | ZADN:ETM-08 | 12.13 | 10.4 | 25.38 | 4.9 | 71.5 | 0.297 | 0.306 |
| Ex2 -9 | ZAD N | ZADN:ETM-09 | 12.19 | 10.4 | 25.77 | 4.9 | 72.4 | 0.296 | 0.307 |
| Ex2 -10 | ETM -05 | ZADN:ETM-01 | 12.01 | 10.4 | 25.71 | 4.9 | 72.3 | 0.299 | 0.310 |
| Ex2 -11 | ETM -05 | ZADN:ETM-02 | 12.08 | 10.4 | 25.56 | 5.0 | 71.9 | 0.296 | 0.305 |
| Ex2 -12 | ETM -05 | ZADN:ETM-03 | 12.17 | 10.5 | 25.72 | 4.9 | 72.4 | 0.298 | 0.309 |
| Ex2 -13 | ETM -05 | ZADN:ETM-04 | 12.12 | 10.4 | 25.68 | 4.9 | 72.2 | 0.297 | 0.308 |
| Ex2 -14 | ETM -05 | ZADN:ETM-05 | 12.08 | 10.5 | 25.48 | 4.9 | 71.8 | 0.297 | 0.307 |
| Ex2 -15 | ETM -05 | ZADN:ETM-06 | 12.16 | 10.4 | 25.42 | 4.9 | 71.5 | 0.296 | 0.306 |
| Ex2 -16 | ETM -05 | ZADN:ETM-07 | 12.10 | 10.5 | 25.54 | 4.9 | 72.0 | 0.299 | 0.308 |
| Ex2 -17 | ETM -05 | ZADN:ETM-08 | 12.10 | 10.3 | 25.58 | 4.9 | 71.9 | 0.297 | 0.308 |
| Ex2 -18 | ETM -05 | ZADN:ETM-09 | 12.02 | 10.4 | 25.76 | 4.9 | 72.4 | 0.298 | 0.310 |
| Ex2 -19 | ETM -06 | ZADN:ETM-01 | 12.11 | 10.5 | 25.74 | 4.9 | 72.4 | 0.298 | 0.309 |
| Ex2 -20 | ETM -06 | ZADN:ETM-02 | 12.16 | 10.5 | 25.77 | 4.9 | 72.5 | 0.298 | 0.309 |
| Ex2 -21 | ETM -06 | ZADN:ETM-03 | 12.20 | 10.5 | 25.72 | 4.9 | 72.4 | 0.298 | 0.308 |
| Ex2 -22 | ETM -06 | ZADN:ETM-04 | 12.05 | 10.4 | 25.39 | 4.9 | 71.5 | 0.297 | 0.307 |
| Ex2 -23 | ETM -06 | ZADN:ETM-05 | 12.05 | 10.3 | 25.43 | 4.9 | 71.5 | 0.295 | 0.305 |
| Ex2 -24 | ETM -06 | ZADN:ETM-06 | 12.19 | 10.4 | 25.83 | 4.9 | 72.5 | 0.296 | 0.308 |
| Ex2 -25 | ETM -06 | ZADN:ETM-07 | 12.07 | 10.4 | 25.78 | 4.9 | 72.4 | 0.298 | 0.310 |
| Ex2 -26 | ETM -06 | ZADN:ETM-08 | 12.15 | 10.3 | 25.68 | 4.9 | 72.1 | 0.298 | 0.309 |
| Ex2 -27 | ETM -06 | ZADN:ETM-09 | 12.10 | 10.5 | 25.55 | 4.9 | 72.0 | 0.298 | 0.307 |
| Ex2 -28 | ETM -07 | ZADN:ETM-01 | 12.01 | 10.4 | 25.47 | 4.9 | 71.6 | 0.297 | 0.308 |
| Ex2 -29 | ETM -07 | ZADN:ETM-02 | 12.16 | 10.5 | 25.95 | 4.9 | 73.0 | 0.298 | 0.309 |
| Ex2 -30 | ETM -07 | ZADN:ETM-03 | 12.07 | 10.4 | 25.52 | 4.9 | 71.8 | 0.296 | 0.306 |
| Ex2 -31 | ETM -07 | ZADN:ETM-04 | 12.02 | 10.5 | 25.72 | 4.9 | 72.3 | 0.299 | 0.309 |
| Ex2 -32 | ETM -07 | ZADN:ETM-05 | 12.11 | 10.5 | 25.55 | 4.9 | 71.9 | 0.298 | 0.308 |
| Ex2 -33 | ETM -07 | ZADN:ETM-06 | 12.04 | 10.4 | 25.45 | 4.9 | 71.6 | 0.297 | 0.307 |
| Ex2 -34 | ETM -07 | ZADN:ETM-07 | 12.08 | 10.5 | 25.69 | 4.9 | 72.4 | 0.298 | 0.308 |
| Ex2 -35 | ETM -07 | ZADN:ETM-08 | 12.06 | 10.5 | 25.67 | 4.9 | 72.2 | 0.298 | 0.308 |
| Ex2 -36 | ETM -07 | ZADN:ETM-09 | 12.18 | 10.3 | 25.51 | 4.9 | 71.7 | 0.296 | 0.307 |
| Ex2 -37 | ETM -08 | ZADN:ETM-01 | 12.16 | 10.4 | 25.77 | 4.9 | 72.3 | 0.297 | 0.309 |
| Ex2 -38 | ETM -08 | ZADN:ETM-02 | 12.15 | 10.4 | 25.57 | 4.9 | 72.0 | 0.298 | 0.308 |
| Ex2 -39 | ETM -08 | ZADN:ETM-03 | 12.08 | 10.5 | 25.58 | 5.0 | 72.1 | 0.296 | 0.306 |
| Ex2 -40 | ETM -08 | ZADN:ETM-04 | 12.05 | 10.6 | 25.91 | 4.9 | 72.9 | 0.299 | 0.310 |
| Ex2 -41 | ETM -08 | ZADN:ETM-05 | 12.14 | 10.4 | 25.71 | 4.9 | 72.2 | 0.298 | 0.309 |
| Ex2 -42 | ETM -08 | ZADN:ETM-06 | 12.09 | 10.4 | 25.66 | 4.9 | 72.2 | 0.298 | 0.309 |
| Ex2 -43 | ETM -08 | ZADN:ETM-07 | 12.04 | 10.4 | 25.60 | 4.9 | 71.9 | 0.297 | 0.308 |
| Ex2 -44 | ETM -08 | ZADN:ETM-08 | 12.11 | 10.5 | 25.90 | 5.0 | 72.9 | 0.297 | 0.307 |
| Ex2 -45 | ETM -08 | ZADN:ETM-09 | 12.13 | 10.5 | 25.71 | 4.9 | 72.4 | 0.299 | 0.309 |

As can be seen from Table 2, the white color coordinates are improved in the second experimental example group.

The driving voltage, red light emission efficiency, green light emission efficiency, blue light emission efficiency, and white light emission efficiency of all three stacks were evaluated at a current density of 10 mA/cm², and the value of white color coordinates in the white subpixel after passing through the microlens array on the light emitting side was evaluated.

As can be seen from Table 2, at least one of the first electron transport layer ETL1 125 and the third electron transport layer ETL3 144 of the first and second blue stacks in the second experimental example group (Ex2-1 to Ex2-45) includes the first material (C1: ETM-01 to ETM-60) in Formula 1. In particular, all CIEx values of the evaluated white color coordinates are 0.299 or less and all CIEy values thereof are 0.310 or less, indicating an improvement in white color coordinates. This is considered to be due to the improved blue light emission efficiency. Substantially, compared with Tables 1 and 2, it can be seen that blue light emission efficiency (B Efficiency) in the second experimental group (Ex2-1 to Ex2-45) is increased by 0.3 Cd/A on average, compared to the first experimental example group (Ex1-1 to Ex1-25).

Hereinafter, the significance of the light emitting device of the present disclosure having improved blue light emission efficiency in the light emitting display device using the microlens array will be determined through comparison with the third experimental example (Ex3) not using the microlens array.

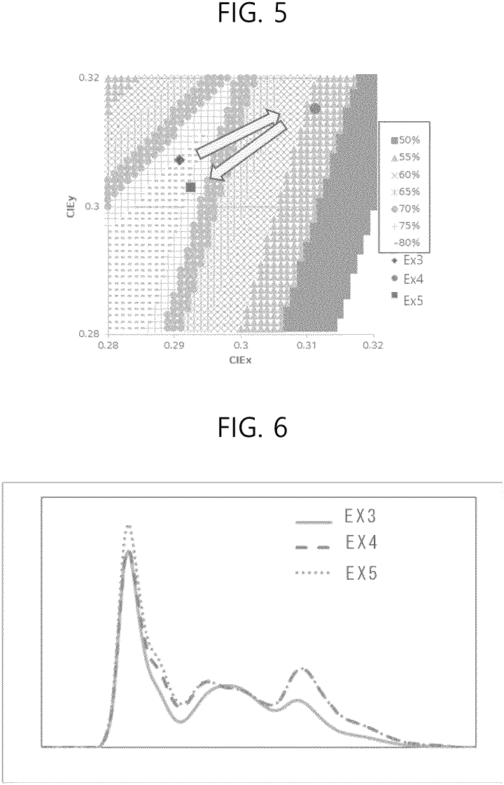

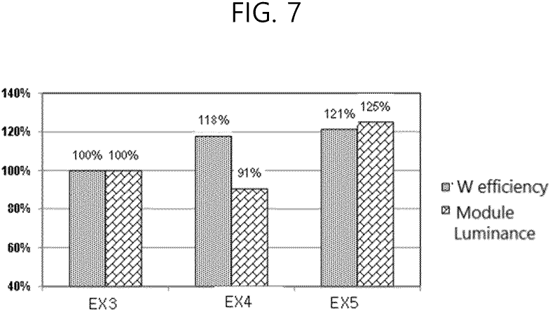

FIG. 5 shows white color coordinates expressed by a light emitting display device including the light emitting devices used in third to fifth experimental examples. FIG. 6 is a graph showing emission spectra of the light emitting devices of third to fifth experimental examples. FIG. 7 is a graph showing white efficiency and module efficiency of light emitting display devices using the light emitting devices of the third to fifth experimental examples.

That is, the fourth experimental example (Ex4) described below corresponds to Ex1-1 of Table 1, all of the electron transport layers used in the blue stacks S1 and S3 include a single material of ZADN, and a microlens having irregularities is provided on the surface of the over-coating layer disposed below the light emitting device having the structure shown in FIG. 4.

In addition, the fifth experimental example (Ex5) corresponds to Ex2-23 of Table 2, and among the electron transport layers used in the blue stacks S1 and S3, the first electron transport layer ETL1 includes ETM-06 and the third electron transport layer (ETL3) includes a mixture of ZADN and ETM-05 at a ratio of 1:1. In addition, a microlens having irregularities is provided on the surface of the over-coating layer located below the light emitting device having the structure of FIG. 4.

In the third experimental example, ZADN is used for all of the electron transport layer material of the blue light emitting layer, like the fourth experimental example, and the structure of the light emitting device of FIG. 4 is formed, but a flat over-coating layer not using the microlens array is used.

In this case, as shown in FIG. 5, the white color coordinates of the light emitting display device of the third experimental example are excellent (about 0.290 and 0.307). However, the micro-lens array is not used, so the light emitting display device to which the third experimental example is applied does not exhibit an improvement in light emission intensity at any wavelength compared to the structure to which the micro-lens array is applied, as shown in FIG. 6, and light emitted from the light emitting device may not be extracted sufficiently.

Meanwhile, in the light emitting display device according to the fourth experimental example, as shown in FIG. 6, although effects of improving green and red light emission efficiency are obtained, there is no improvement for blue wavelengths, so the color coordinates of white, obtained by combining red, green, and blue, are at the level of (0.310, 0314) owing to the high green and red intensities, as shown in FIG. 5.

However, the light emitting display device according to the fifth experimental example including the material of Formula 1 in at least one electron transport layer of the blue stack, as shown in FIG. 5, has the effect of improving the efficiency of green and red light emission, comparable to the fourth experimental example, through the microlens array, and is capable of improving blue light emission efficiency, as shown in FIG. 6 and thus uniformly improving the efficiency in the entire visible light wavelength band, by changing the material for the electron transport layer in the blue stack in the light emitting device.

This will be described with reference to FIG. 7. The white efficiency can be improved using the microlens array in both the fourth and fifth experimental examples, compared to the third experimental example.

However, in the module stage, it is important to have a balance between red, green, and blue in expressing white in consideration of light absorption at the light emitting side, for example by a color filter or a polarizing plate. The data on the right side of each of the third to fifth experimental examples of FIG. 7 is the module luminance. In the fourth experimental example, there is an improvement in the efficiency of the microlens array only in green and red, unlike blue, so the efficiency is 91%, which is lower than 100%, which is the efficiency of the third experimental example.

On the other hand, like the light emitting device of the present disclosure, the light emitting display device according to the fifth experimental example, having a blue stack, to which an electron transport layer including the material of Formula 1 is applied, has a module luminance of 125% that of the third experimental example. It can be seen that the light emitting device alone and a combination of the light emitting device with a microlens array and other color filters or light-absorbing materials can exhibit improved luminance efficiency.

Accordingly, the light emitting display device of the present disclosure is characterized in that a microlens array is used, the light emitting device includes at least two blue stacks and a phosphorescent stack, and at least one electron transport layer of the blue stack is formed of the material of Formula 1.

That is, in the light emitting display device of the present disclosure, in order to compensate for the efficiency of blue, which is not affected by the structure of the microlens array that is used, the material of the electron transport layer in contact with the blue light emitting layer is changed to increase the recombination rate of holes and electrons in the blue light emitting layer and thus improve the external quantum efficiency of blue, thereby balancing the increase in the efficiency of green and red light emission due to the use of the microlens array.

In addition, the blue stack uses a fluorescent material determined in consideration of both efficiency and lifespan, and includes two or more stacks in the light emitting device to obtain a high color temperature level required for white expression. In this case, the blue stack is disposed so as to contact the anode and the cathode. Among them, the electron transport layer in direct contact with the electron injection layer formed of an inorganic material or inorganic compound may further include a material having compatibility with the electron injection layer to prevent an increase in driving voltage and prolong the lifespan thereof.

The light emitting display device according to an embodiment of the present disclosure includes a substrate having a plurality of subpixels, each including a light emitting region and a non-light emitting region, an over-coating layer having irregularities on a surface thereof in at least the light emitting region, and a light emitting device disposed on the surface of the over-coating layer, the light emitting device including an anode and a cathode facing each other, and at least two blue stacks and a phosphorescent stack between the anode and the cathode, wherein each of the blue stacks includes a blue light emitting layer having an emission peak of 420 nm to 480 nm, at least one of the blue stacks is in contact with the blue light emitting layer and includes an electron transport layer including a first material of Formula 1, and the phosphorescent stack includes at least two phosphorescent light emitting layers emitting light having a wavelength longer than that of the blue light emitting layer.

In a light emitting display device, a micro lens, which is a micro-sized light extraction lens, may be applied to improve total reflection of light, related to a phenomenon in which light emitted from a light emitting device is trapped in a substrate. In this case, the external luminous efficiency increases, but the effect is mainly at a wavelength of 480 nm or more, and the light extraction effect is insufficient in the range of 400 to 470 nm, which is the main wavelength region of blue. The improvement attributable to the micro lens array is not great due to the characteristics of the white-light emitting device that requires the light extraction effect improved through the balance between the three primary colors.

The present disclosure aims at overcoming the difference in light extraction efficiency for each color wavelength band by changing the material in the light emitting device. In particular, by using a material having excellent electron transport capability for the electron transport layer in the blue stack, both the overall efficiency and the white light emission efficiency of the light emitting display device can be improved.

The light emitting device and the light emitting display device according to the present disclosure have the following effects.

A microlens array is provided in contact with the light emitting device to maximize or increase emission of light from the light emitting device having a plurality of light emitting layers, while preventing the light from being trapped in the substrate.

In order to compensate for the efficiency of the blue color that is not affected by the structure of the microlens array that is used, the material of the electron transport layer in contact with the blue light emitting layer is changed, so the recombination rate of holes and electrons in the blue light emitting layer can be improved, the external quantum efficiency of blue can be improved, and thus the efficiency of green and red light emission can be improved due to the use of microlens arrays.

The blue stack uses a fluorescent material determined in consideration of both efficiency and lifespan, and includes two or more stacks in the light emitting device to obtain the high color temperature level for white expression. In this case, the blue stack may be disposed so as to contact the anode and the cathode. In particular, the electron transport layer in direct contact with the electron injection layer made of an inorganic material or inorganic compound may further include a material having compatibility with the electron injection layer to prevent an increase in driving voltage and improve lifespan.

The light emitting display device according to an embodiment of the present disclosure includes a substrate having a plurality of subpixels, each including a light emitting region and a non-light emitting region, an over-coating layer having irregularities on a surface thereof in at least the light emitting region, and a light emitting device disposed on the surface of the over-coating layer, the light emitting device including an anode and a cathode facing each other, and at least two blue stacks and a phosphorescent stack between the anode and the cathode, wherein each of the blue stacks includes a blue light emitting layer having an emission peak of 420 nm to 480 nm and an electron transport layer including a first material of Formula 1, the electron transport layer in contact with the blue light emitting layer, and the phosphorescent stack includes at least two phosphorescent light emitting layers emitting light having a wavelength longer than that of the blue light emitting layer.

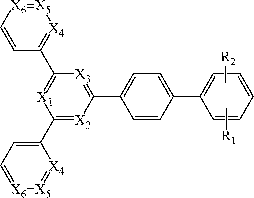

wherein R₁ and R₂ are each independently selected from a cycloalkyl group, an aryl group, a heteroaryl group, and a carbazole group, X₁, X₂ and X₃ are each independently N or CH, and at least one of X₄, X₅ and X₆ is N and the remaining ones are CH.

The phosphorescent stack may include a first phosphorescent light emitting layer having an emission peak of 600 nm to 660 nm, a second phosphorescent light emitting layer having an emission peak of 530 nm to 600 nm, and a third phosphorescent light emitting layer having an emission peak of 500 nm to 530 nm, which are laminated in this order, wherein, among the first to third phosphorescent layers, the first phosphorescent light emitting layer is closest to the anode.

The at least two blue stacks may include a first blue stack in contact with the anode, and a second blue stack in contact with the cathode, and the light emitting display device may further include, as a second material, a compound containing benzimidazole in the second electron transport layer of the second blue stack, unlike the first electron transport layer of the first blue stack.

The first material and the second material in the second electron transport layer may be present at equal amounts.

The light emitting display device may further include an electron injection layer formed of an inorganic material or an inorganic compound between the second electron transport layer and the cathode.

The blue light emitting layer of the first blue stack may have a vertical distance of 100 nm to 300 nm from the anode.

The blue light emitting layer may include a boron-based dopant.

The light emitting display device may further include an electron blocking layer on a surface opposite to each blue light emitting layer, where the electron transport layer is not formed, in the blue stack, and the electron blocking layer is adjacent to a hole transport layer on the surface opposite to the blue light emitting layer.

The light emitting display device may further include a charge generation layer including a stack of an n-type charge generation layer and a p-type charge generation layer between the blue stack and the phosphorescent stack, wherein the n-type charge generation layer is adjacent to the electron transport layer of the adjacent blue stack or the phosphorescent stack, and the p-type charge generation layer is adjacent to the hole transport layer of the blue stack or the phosphorescent stack.

A distance between a top and a bottom of irregularities on the surface of the over-coating layer may be greater than a distance between the anode and the cathode of the light emitting device.

The anode may include a transparent electrode and the cathode may include a reflective electrode.

The substrate may include at least one thin film transistor in a non-light emitting region of the subpixel, and a color filter in the light emitting region of the subpixel.

Light generated from the light emitting device may be emitted through the irregularities on the surface of the over-coating layer, the color filter, and the substrate.

It will be apparent to those skilled in the art that various modifications and variations can be made in the present invention without departing from the spirit or scope of the invention. Thus, it is intended that the present disclosure covers such modifications and variations thereto, provided they fall within the scope of the appended claims and their equivalents.

What is claimed is:

1. A light emitting display device comprising:
a substrate having a plurality of subpixels, each comprising a light emitting region and a non-light emitting region;
an over-coating layer having irregularities on a surface thereof in at least the light emitting region; and
a light emitting device disposed on the surface of the over-coating layer, the light emitting device comprising an anode and a cathode facing each other, and at least two blue stacks and a phosphorescent stack between the anode and the cathode,
wherein each of the blue stacks comprises a blue light emitting layer having an emission peak of 420 nm to 480 nm,
wherein at least one of the blue stacks comprises an electron transport layer including a first material of Formula 1, the electron transport layer being in contact with the blue light emitting layer, and 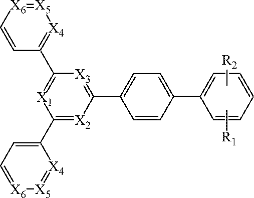
wherein R₁ and R₂ are each independently selected from a cycloalkyl group, an aryl group, a heteroaryl group, and a carbazole group;
X₁, X₂ and X₃ are each independently N or CH; and
at least one of X₄, X₅, and X₆ is N and the remaining ones are CH,
wherein the phosphorescent stack comprises at least two phosphorescent light emitting layers configured to emit light having wavelengths longer than that of the blue light emitting layer.

2. The light emitting display device according to claim 1, wherein the phosphorescent stack comprises a first phosphorescent light emitting layer having an emission peak of 600 nm to 660 nm, a second phosphorescent light emitting layer having an emission peak of 530 nm to 600 nm, and a third phosphorescent light emitting layer having an emission peak of 500 nm to 530 nm, which are laminated in this order,
wherein, among the first phosphorescent light emitting layer, the second phosphorescent light emitting layer, and the third phosphorescent light emitting layer, the first phosphorescent light emitting layer is closest to the anode.

3. The light emitting display device according to claim 1, wherein the at least two blue stacks comprise:
a first blue stack in contact with the anode; and
a second blue stack in contact with the cathode,
the light emitting display device further comprises, as a second material, a compound including benzimidazole in a second electron transport layer of the second blue stack, than a first electron transport layer of the first blue stack.

4. The light emitting display device according to claim 3, wherein the first material and the second material in the second electron transport layer are present at equal amounts.

5. The light emitting display device according to claim 3, further comprising an electron injection layer formed of an inorganic material or an inorganic compound between the second electron transport layer and the cathode.

6. The light emitting display device according to claim 3, wherein the blue light emitting layer of the first blue stack has a vertical distance of 100 nm to 300 nm from the anode.

7. The light emitting display device according to claim 1, wherein the blue light emitting layer comprises a boron-based dopant.

8. The light emitting display device according to claim 1, further comprising an electron blocking layer contacting a surface of each blue light emitting layer, and
the electron blocking layer is adjacent to a hole transport layer.

9. The light emitting display device according to claim 1, further comprising a charge generation layer comprising an n-type charge generation layer and a p-type charge generation layer between the at least one of the blue stacks and the phosphorescent stack,
wherein the n-type charge generation layer is adjacent to the electron transport layer or an electron transport layer of the phosphorescent stack, and the p-type charge generation layer is adjacent to a hole transport layer of the at least one of the blue stacks or the phosphorescent stack.

10. The light emitting display device according to claim 1, wherein a distance between a top and a bottom of the irregularities on the surface of the over-coating layer is greater than a distance between the anode and the cathode of the light emitting device.

11. The light emitting display device according to claim 1, wherein the substrate comprises at least one thin film transistor in the non-light emitting region of the subpixel, and a color filter in the light emitting region of the subpixel.

12. The light emitting display device according to claim 11,
wherein the anode comprises a transparent electrode and the cathode comprises a reflective electrode and
wherein light generated from the light emitting device is emitted through the irregularities on the surface of the over-coating layer, the color filter, and the substrate.

13. The light emitting display device according to claim 1, wherein Formula 1 is given by: 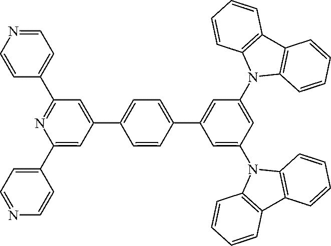 [SMILES: c1ccc2c(c1)c1ccccc1n2-c1cc(-c2ccc(-c3cc(-c4ccncc4)nc(-c4ccncc4)c3)cc2)cc(-n2c3ccccc3c3ccccc32)c1] 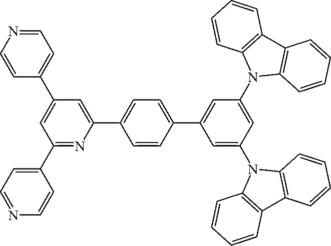 [SMILES: c1ccc2c(c1)c1ccccc1n2-c1cc(-c2ccc(-c3cc(-c4ccncc4)cc(-c4ccncc4)n3)cc2)cc(-n2c3ccccc3c3ccccc32)c1] 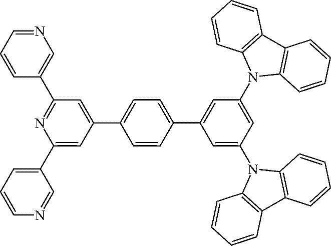 [SMILES: c1cncc(-c2cc(-c3ccc(-c4cc(-n5c6ccccc6c6ccccc65)cc(-n5c6ccccc6c6ccccc65)c4)cc3)cc(-c3cccnc3)n2)c1] 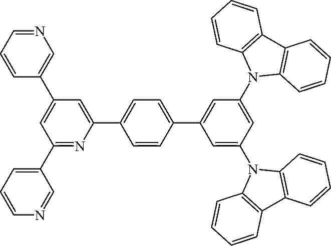 [SMILES: c1cncc(-c2cc(-c3ccc(-c4cc(-n5c6ccccc6c6ccccc65)cc(-n5c6ccccc6c6ccccc65)c4)cc3)nc(-c3cccnc3)c2)c1] 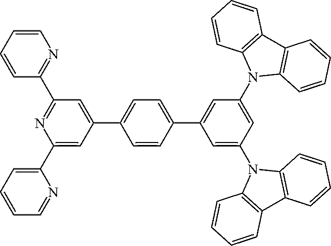 [SMILES: c1ccc(-c2cc(-c3ccc(-c4cc(-n5c6ccccc6c6ccccc65)cc(-n5c6ccccc6c6ccccc65)c4)cc3)cc(-c3ccccn3)n2)nc1] 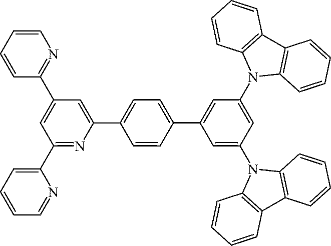 [SMILES: c1ccc(-c2cc(-c3ccc(-c4cc(-n5c6ccccc6c6ccccc65)cc(-n5c6ccccc6c6ccccc65)c4)cc3)nc(-c3ccccn3)c2)nc1] 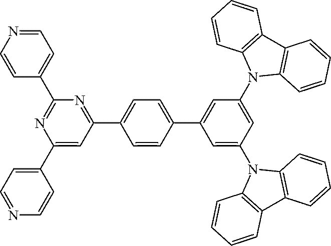 [SMILES: c1ccc2c(c1)c1ccccc1n2-c1cc(-c2ccc(-c3cc(-c4ccncc4)nc(-c4ccncc4)n3)cc2)cc(-n2c3ccccc3c3ccccc32)c1] 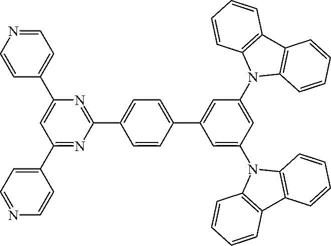 [SMILES: c1ccc2c(c1)c1ccccc1n2-c1cc(-c2ccc(-c3nc(-c4ccncc4)cc(-c4ccncc4)n3)cc2)cc(-n2c3ccccc3c3ccccc32)c1] 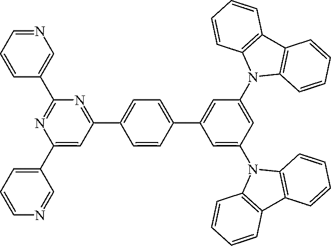 [SMILES: c1cncc(-c2cc(-c3ccc(-c4cc(-n5c6ccccc6c6ccccc65)cc(-n5c6ccccc6c6ccccc65)c4)cc3)nc(-c3cccnc3)n2)c1] 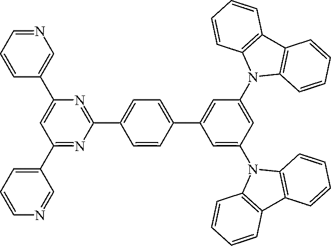 [SMILES: c1cncc(-c2cc(-c3cccnc3)nc(-c3ccc(-c4cc(-n5c6ccccc6c6ccccc65)cc(-n5c6ccccc6c6ccccc65)c4)cc3)n2)c1] 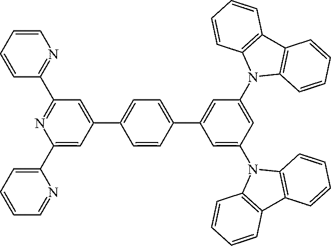 [SMILES: c1ccc(-c2cc(-c3ccc(-c4cc(-n5c6ccccc6c6ccccc65)cc(-n5c6ccccc6c6ccccc65)c4)cc3)cc(-c3ccccn3)n2)nc1] 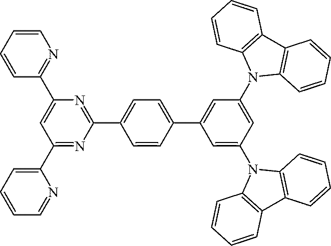 [SMILES: c1ccc(-c2cc(-c3ccccn3)nc(-c3ccc(-c4cc(-n5c6ccccc6c6ccccc65)cc(-n5c6ccccc6c6ccccc65)c4)cc3)n2)nc1] 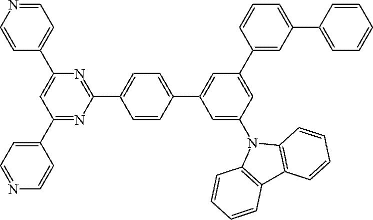 [SMILES: c1ccc(-c2cccc(-c3cc(-c4ccc(-c5nc(-c6ccncc6)cc(-c6ccncc6)n5)cc4)cc(-n4c5ccccc5c5ccccc54)c3)c2)cc1] 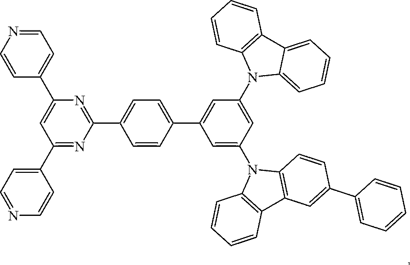 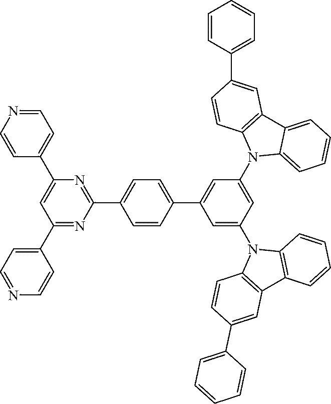 [SMILES: c1ccc(-c2ccc3c(c2)c2ccccc2n3-c2cc(-c3ccc(-c4nc(-c5ccncc5)cc(-c5ccncc5)n4)cc3)cc(-n3c4ccccc4c4cc(-c5ccccc5)ccc43)c2)cc1] 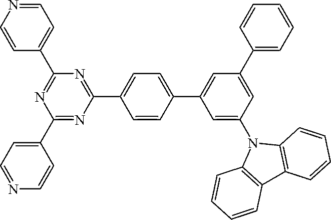 [SMILES: c1ccc(-c2cc(-c3ccc(-c4nc(-c5ccncc5)nc(-c5ccncc5)n4)cc3)cc(-n3c4ccccc4c4ccccc43)c2)cc1] 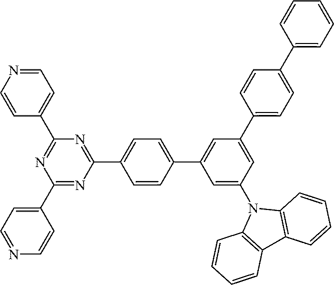 [SMILES: c1ccc(-c2ccc(-c3cc(-c4ccc(-c5nc(-c6ccncc6)nc(-c6ccncc6)n5)cc4)cc(-n4c5ccccc5c5ccccc54)c3)cc2)cc1] 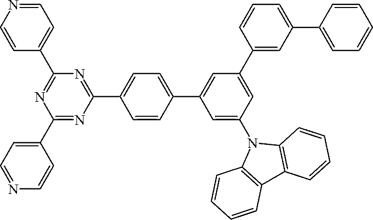 [SMILES: c1ccc(-c2cccc(-c3cc(-c4ccc(-c5nc(-c6ccncc6)nc(-c6ccncc6)n5)cc4)cc(-n4c5ccccc5c5ccccc54)c3)c2)cc1] 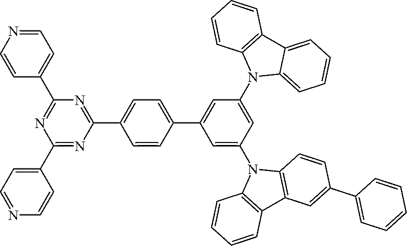 [SMILES: c1ccc(-c2ccc3c(c2)c2ccccc2n3-c2cc(-c3ccc(-c4nc(-c5ccncc5)nc(-c5ccncc5)n4)cc3)cc(-n3c4ccccc4c4ccccc43)c2)cc1] 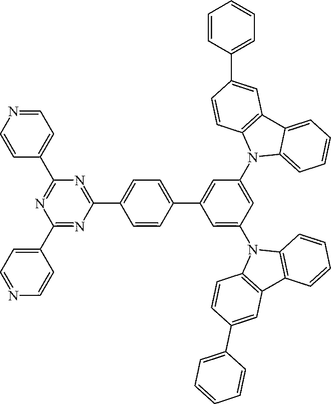 [SMILES: c1ccc(-c2ccc3c(c2)c2ccccc2n3-c2cc(-c3ccc(-c4nc(-c5ccncc5)nc(-c5ccncc5)n4)cc3)cc(-n3c4ccccc4c4cc(-c5ccccc5)ccc43)c2)cc1] 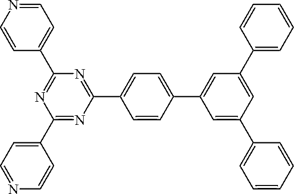 [SMILES: c1ccc(-c2cc(-c3ccccc3)cc(-c3ccc(-c4nc(-c5ccncc5)nc(-c5ccncc5)n4)cc3)c2)cc1] 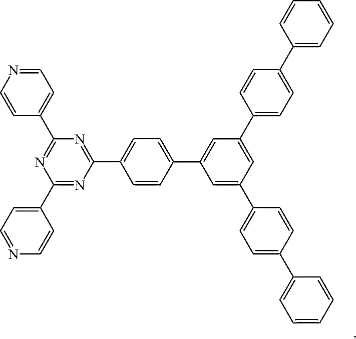 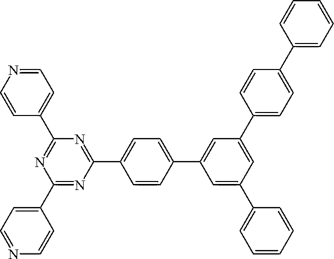 [SMILES: c1ccc(-c2ccc(-c3cc(-c4ccccc4)cc(-c4ccc(-c5nc(-c6ccncc6)nc(-c6ccncc6)n5)cc4)c3)cc2)cc1] 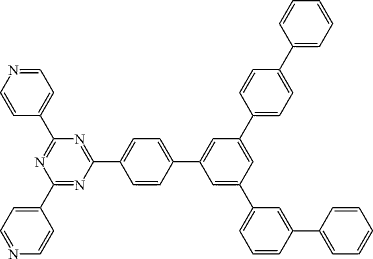 [SMILES: c1ccc(-c2ccc(-c3cc(-c4ccc(-c5nc(-c6ccncc6)nc(-c6ccncc6)n5)cc4)cc(-c4cccc(-c5ccccc5)c4)c3)cc2)cc1] 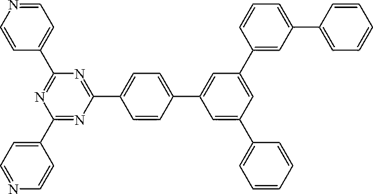 [SMILES: c1ccc(-c2cccc(-c3cc(-c4ccccc4)cc(-c4ccc(-c5nc(-c6ccncc6)nc(-c6ccncc6)n5)cc4)c3)c2)cc1] 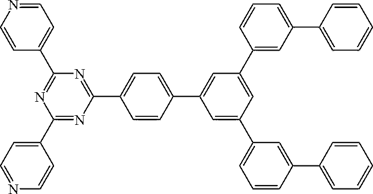 [SMILES: c1ccc(-c2cccc(-c3cc(-c4ccc(-c5nc(-c6ccncc6)nc(-c6ccncc6)n5)cc4)cc(-c4cccc(-c5ccccc5)c4)c3)c2)cc1] 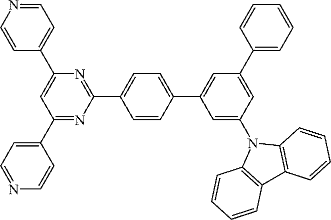 [SMILES: c1ccc(-c2cc(-c3ccc(-c4nc(-c5ccncc5)cc(-c5ccncc5)n4)cc3)cc(-n3c4ccccc4c4ccccc43)c2)cc1] 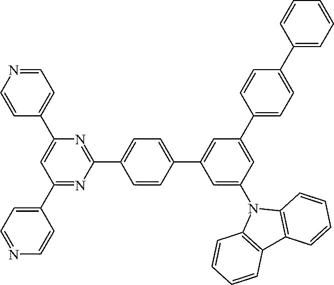 [SMILES: c1ccc(-c2ccc(-c3cc(-c4ccc(-c5nc(-c6ccncc6)cc(-c6ccncc6)n5)cc4)cc(-n4c5ccccc5c5ccccc54)c3)cc2)cc1] 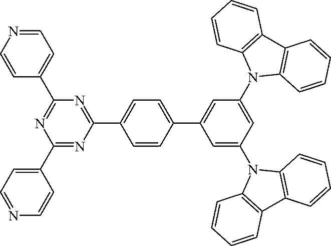 [SMILES: c1ccc2c(c1)c1ccccc1n2-c1cc(-c2ccc(-c3nc(-c4ccncc4)nc(-c4ccncc4)n3)cc2)cc(-n2c3ccccc3c3ccccc32)c1] 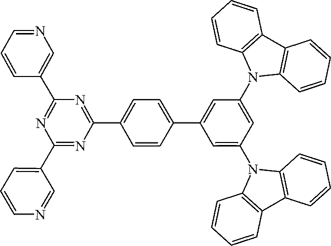 [SMILES: c1cncc(-c2nc(-c3ccc(-c4cc(-n5c6ccccc6c6ccccc65)cc(-n5c6ccccc6c6ccccc65)c4)cc3)nc(-c3cccnc3)n2)c1] 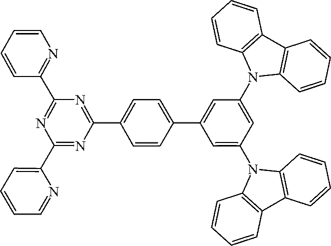 [SMILES: c1ccc(-c2nc(-c3ccc(-c4cc(-n5c6ccccc6c6ccccc65)cc(-n5c6ccccc6c6ccccc65)c4)cc3)nc(-c3ccccn3)n2)nc1] 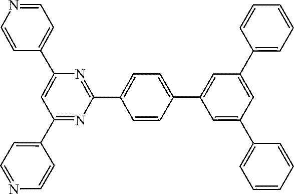 [SMILES: c1ccc(-c2cc(-c3ccccc3)cc(-c3ccc(-c4nc(-c5ccncc5)cc(-c5ccncc5)n4)cc3)c2)cc1] 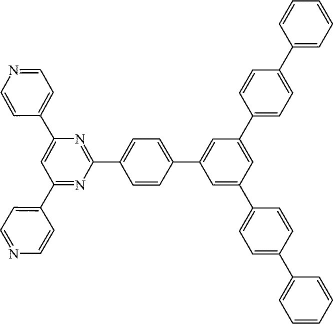 [SMILES: c1ccc(-c2ccc(-c3cc(-c4ccc(-c5ccccc5)cc4)cc(-c4ccc(-c5nc(-c6ccncc6)cc(-c6ccncc6)n5)cc4)c3)cc2)cc1] 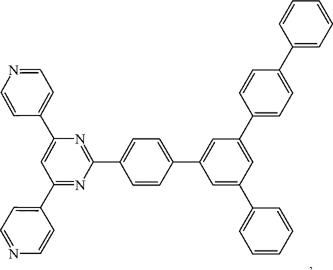 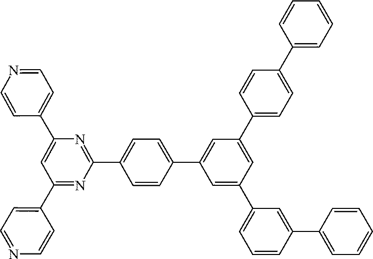 [SMILES: c1ccc(-c2ccc(-c3cc(-c4ccc(-c5nc(-c6ccncc6)cc(-c6ccncc6)n5)cc4)cc(-c4cccc(-c5ccccc5)c4)c3)cc2)cc1] 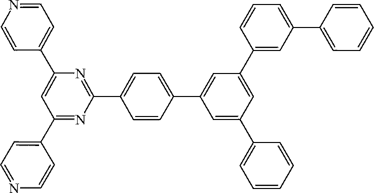 [SMILES: c1ccc(-c2cccc(-c3cc(-c4ccccc4)cc(-c4ccc(-c5nc(-c6ccncc6)cc(-c6ccncc6)n5)cc4)c3)c2)cc1] 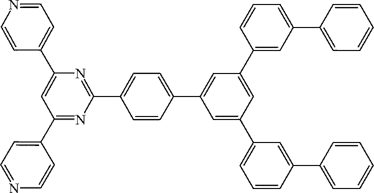 [SMILES: c1ccc(-c2cccc(-c3cc(-c4ccc(-c5nc(-c6ccncc6)cc(-c6ccncc6)n5)cc4)cc(-c4cccc(-c5ccccc5)c4)c3)c2)cc1] 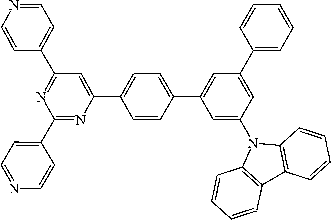 [SMILES: c1ccc(-c2cc(-c3ccc(-c4cc(-c5ccncc5)nc(-c5ccncc5)n4)cc3)cc(-n3c4ccccc4c4ccccc43)c2)cc1] 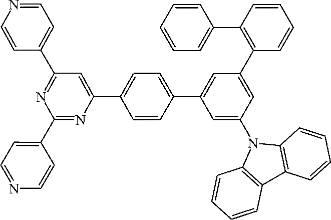 [SMILES: c1ccc(-c2ccccc2-c2cc(-c3ccc(-c4cc(-c5ccncc5)nc(-c5ccncc5)n4)cc3)cc(-n3c4ccccc4c4ccccc43)c2)cc1] 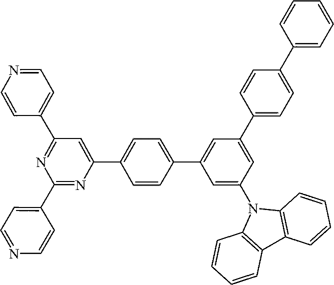 [SMILES: c1ccc(-c2ccc(-c3cc(-c4ccc(-c5cc(-c6ccncc6)nc(-c6ccncc6)n5)cc4)cc(-n4c5ccccc5c5ccccc54)c3)cc2)cc1] 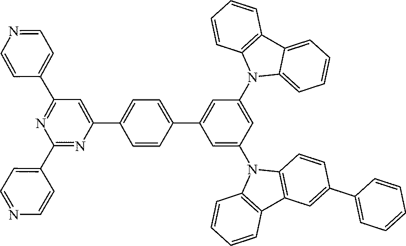 [SMILES: c1ccc(-c2ccc3c(c2)c2ccccc2n3-c2cc(-c3ccc(-c4cc(-c5ccncc5)nc(-c5ccncc5)n4)cc3)cc(-n3c4ccccc4c4ccccc43)c2)cc1] 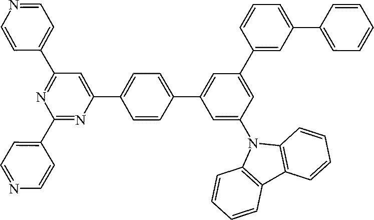 [SMILES: c1ccc(-c2cccc(-c3cc(-c4ccc(-c5cc(-c6ccncc6)nc(-c6ccncc6)n5)cc4)cc(-n4c5ccccc5c5ccccc54)c3)c2)cc1] 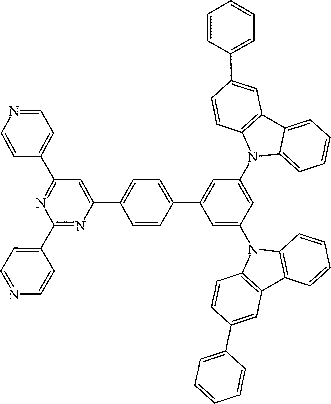 [SMILES: c1ccc(-c2ccc3c(c2)c2ccccc2n3-c2cc(-c3ccc(-c4cc(-c5ccncc5)nc(-c5ccncc5)n4)cc3)cc(-n3c4ccccc4c4cc(-c5ccccc5)ccc43)c2)cc1] 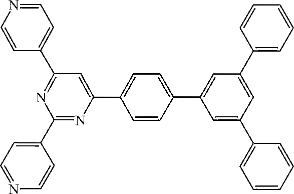 [SMILES: c1ccc(-c2cc(-c3ccccc3)cc(-c3ccc(-c4cc(-c5ccncc5)nc(-c5ccncc5)n4)cc3)c2)cc1] 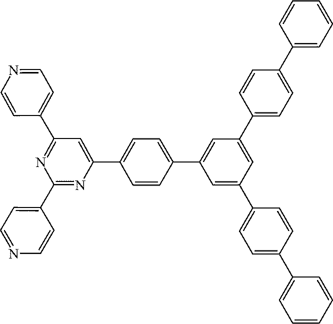 [SMILES: c1ccc(-c2ccc(-c3cc(-c4ccc(-c5ccccc5)cc4)cc(-c4ccc(-c5cc(-c6ccncc6)nc(-c6ccncc6)n5)cc4)c3)cc2)cc1] 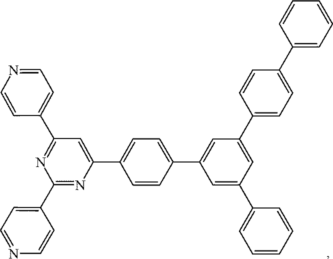 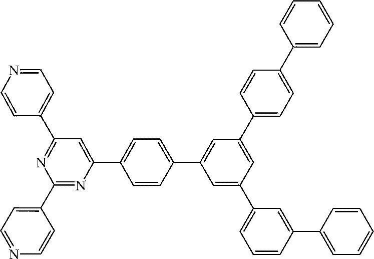 [SMILES: c1ccc(-c2ccc(-c3cc(-c4ccc(-c5cc(-c6ccncc6)nc(-c6ccncc6)n5)cc4)cc(-c4cccc(-c5ccccc5)c4)c3)cc2)cc1] 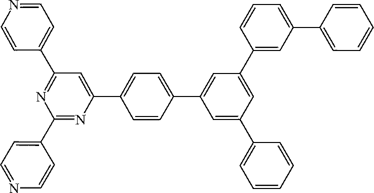 [SMILES: c1ccc(-c2cccc(-c3cc(-c4ccccc4)cc(-c4ccc(-c5cc(-c6ccncc6)nc(-c6ccncc6)n5)cc4)c3)c2)cc1] 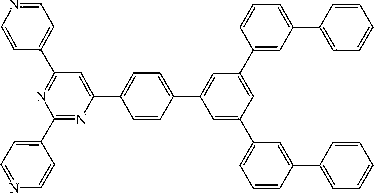 [SMILES: c1ccc(-c2cccc(-c3cc(-c4ccc(-c5cc(-c6ccncc6)nc(-c6ccncc6)n5)cc4)cc(-c4cccc(-c5ccccc5)c4)c3)c2)cc1] 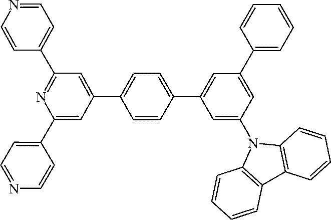 [SMILES: c1ccc(-c2cc(-c3ccc(-c4cc(-c5ccncc5)nc(-c5ccncc5)c4)cc3)cc(-n3c4ccccc4c4ccccc43)c2)cc1] 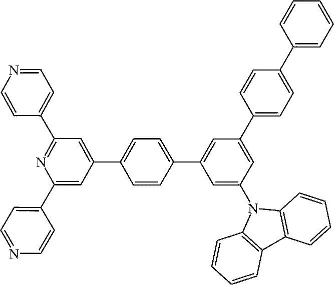 [SMILES: c1ccc(-c2ccc(-c3cc(-c4ccc(-c5cc(-c6ccncc6)nc(-c6ccncc6)c5)cc4)cc(-n4c5ccccc5c5ccccc54)c3)cc2)cc1] 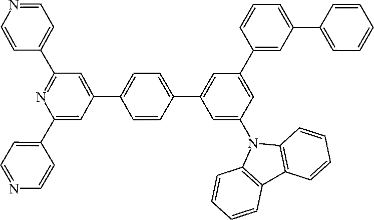 [SMILES: c1ccc(-c2cccc(-c3cc(-c4ccc(-c5cc(-c6ccncc6)nc(-c6ccncc6)c5)cc4)cc(-n4c5ccccc5c5ccccc54)c3)c2)cc1] 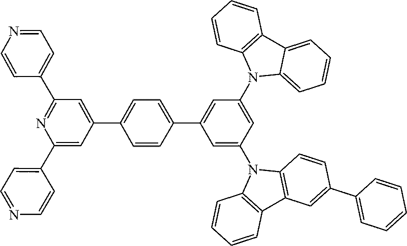 [SMILES: c1ccc(-c2ccc3c(c2)c2ccccc2n3-c2cc(-c3ccc(-c4cc(-c5ccncc5)nc(-c5ccncc5)c4)cc3)cc(-n3c4ccccc4c4ccccc43)c2)cc1] 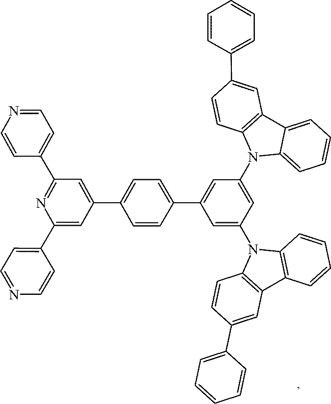 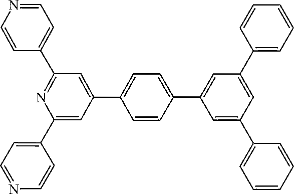 [SMILES: c1ccc(-c2cc(-c3ccccc3)cc(-c3ccc(-c4cc(-c5ccncc5)nc(-c5ccncc5)c4)cc3)c2)cc1] 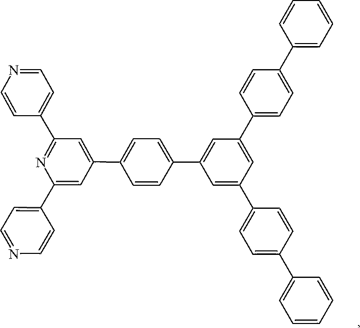 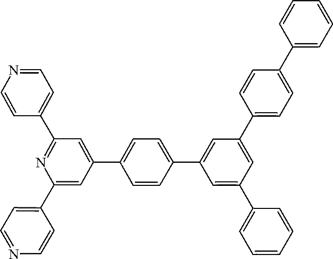 [SMILES: c1ccc(-c2ccc(-c3cc(-c4ccccc4)cc(-c4ccc(-c5cc(-c6ccncc6)nc(-c6ccncc6)c5)cc4)c3)cc2)cc1] 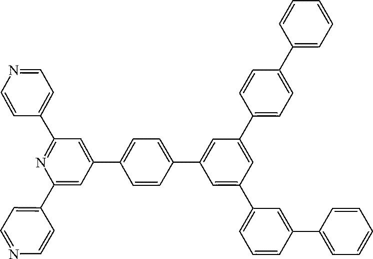 [SMILES: c1ccc(-c2ccc(-c3cc(-c4ccc(-c5cc(-c6ccncc6)nc(-c6ccncc6)c5)cc4)cc(-c4cccc(-c5ccccc5)c4)c3)cc2)cc1] 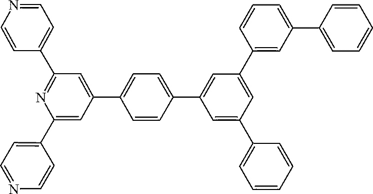 [SMILES: c1ccc(-c2cccc(-c3cc(-c4ccccc4)cc(-c4ccc(-c5cc(-c6ccncc6)nc(-c6ccncc6)c5)cc4)c3)c2)cc1] 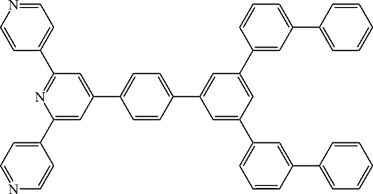 [SMILES: c1ccc(-c2cccc(-c3cc(-c4ccc(-c5cc(-c6ccncc6)nc(-c6ccncc6)c5)cc4)cc(-c4cccc(-c5ccccc5)c4)c3)c2)cc1]
.

14. A light emitting display device comprising:
a substrate having a plurality of subpixels, each comprising a light emitting region and a non-light emitting region;
an over-coating layer having a plurality of concave portions and convex portions on a surface of the over-coating layer in a light emitting region for a subpixel; and
a white light emitting device disposed on the surface of the over-coating layer for the subpixel, the white light emitting device comprising an anode, an organic layer on the anode, and a cathode on the organic layer, wherein the anode, the organic layer, and the cathode have a plurality of concave portions and convex portions corresponding to the concave portions and the convex portions of the over-coating layer respectively, the organic layer further including:
one or more blue stacks for emitting blue light wherein at least a first blue stack comprises an electron transport layer including a first material of Formula 1; and
one or more phosphorescent stacks for emitting light at a higher wavelength than the one or more blue stacks,
wherein Formula 1 is: 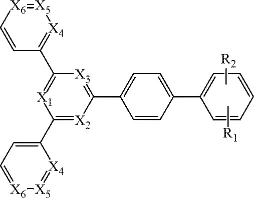
wherein R₁ and R₂ are each independently selected from a cycloalkyl group, an aryl group, a heteroaryl group, and a carbazole group;
X₁, X₂ and X₃ are each independently N or CH; and
at least one of X₄, X₅ and X₆ is N and the remaining ones are CH.

15. The light emitting display device according to claim 14, wherein the one or more blue stacks comprises a second blue stack, and wherein the second blue stack comprises the first material and a second material including a benzimidazole group.

16. The light emitting display device according to claim 15, wherein the first blue stack is closer to the anode than the second blue stack and the second blue stack is closer to the cathode than the first blue stack.

17. The light emitting display device according to claim 15, wherein the second material is given by Formula 2: 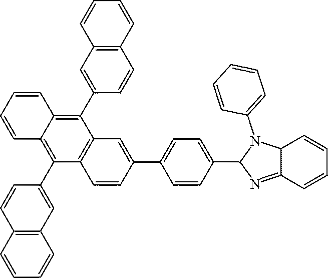 [SMILES: C1=CC2=NC(c3ccc(-c4ccc5c(-c6ccc7ccccc7c6)c6ccccc6c(-c6ccc7ccccc7c6)c5c4)cc3)N(c3ccccc3)C2C=C1]
.

18. The light emitting display device according to claim 14, wherein a distance between a top portion and a bottom portion of a concave portion of the over-coating layer is greater than a thickness of the organic layer.

19. The light emitting display device according to claim 14, wherein the one or more phosphorescent stacks includes at least a red light emitting layer and a green light emitting layer.

20. The light emitting display device according to claim 14, wherein Formula 1 is given by: 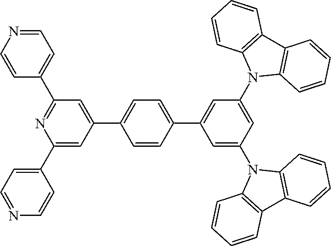 [SMILES: c1ccc2c(c1)c1ccccc1n2-c1cc(-c2ccc(-c3cc(-c4ccncc4)nc(-c4ccncc4)c3)cc2)cc(-n2c3ccccc3c3ccccc32)c1] 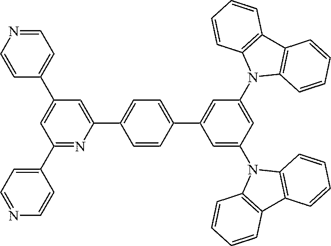 [SMILES: c1ccc2c(c1)c1ccccc1n2-c1cc(-c2ccc(-c3cc(-c4ccncc4)cc(-c4ccncc4)n3)cc2)cc(-n2c3ccccc3c3ccccc32)c1] 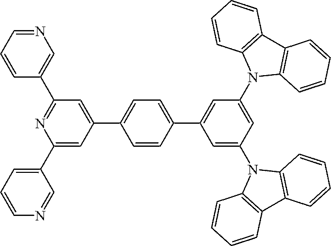 [SMILES: c1cncc(-c2cc(-c3ccc(-c4cc(-n5c6ccccc6c6ccccc65)cc(-n5c6ccccc6c6ccccc65)c4)cc3)cc(-c3cccnc3)n2)c1] 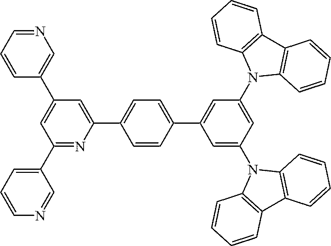 [SMILES: c1cncc(-c2cc(-c3ccc(-c4cc(-n5c6ccccc6c6ccccc65)cc(-n5c6ccccc6c6ccccc65)c4)cc3)nc(-c3cccnc3)c2)c1] 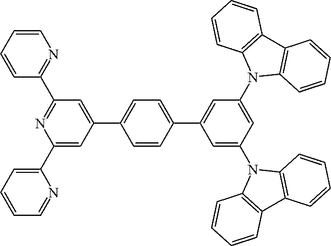 [SMILES: c1ccc(-c2cc(-c3ccc(-c4cc(-n5c6ccccc6c6ccccc65)cc(-n5c6ccccc6c6ccccc65)c4)cc3)cc(-c3ccccn3)n2)nc1] 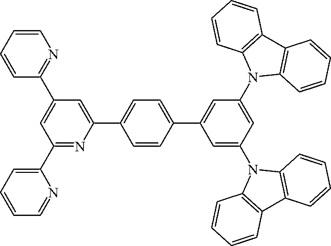 [SMILES: c1ccc(-c2cc(-c3ccc(-c4cc(-n5c6ccccc6c6ccccc65)cc(-n5c6ccccc6c6ccccc65)c4)cc3)nc(-c3ccccn3)c2)nc1] 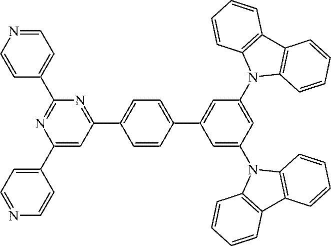 [SMILES: c1ccc2c(c1)c1ccccc1n2-c1cc(-c2ccc(-c3cc(-c4ccncc4)nc(-c4ccncc4)n3)cc2)cc(-n2c3ccccc3c3ccccc32)c1] 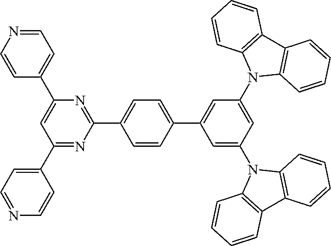 [SMILES: c1ccc2c(c1)c1ccccc1n2-c1cc(-c2ccc(-c3nc(-c4ccncc4)cc(-c4ccncc4)n3)cc2)cc(-n2c3ccccc3c3ccccc32)c1] 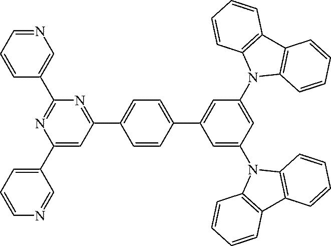 [SMILES: c1cncc(-c2cc(-c3ccc(-c4cc(-n5c6ccccc6c6ccccc65)cc(-n5c6ccccc6c6ccccc65)c4)cc3)nc(-c3cccnc3)n2)c1] 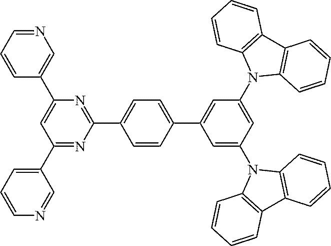 [SMILES: c1cncc(-c2cc(-c3cccnc3)nc(-c3ccc(-c4cc(-n5c6ccccc6c6ccccc65)cc(-n5c6ccccc6c6ccccc65)c4)cc3)n2)c1] 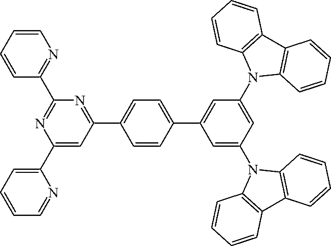 [SMILES: c1ccc(-c2cc(-c3ccc(-c4cc(-n5c6ccccc6c6ccccc65)cc(-n5c6ccccc6c6ccccc65)c4)cc3)nc(-c3ccccn3)n2)nc1] 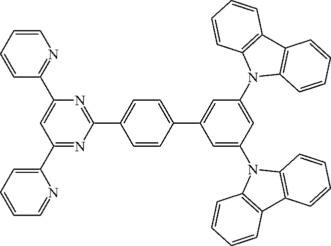 [SMILES: c1ccc(-c2cc(-c3ccccn3)nc(-c3ccc(-c4cc(-n5c6ccccc6c6ccccc65)cc(-n5c6ccccc6c6ccccc65)c4)cc3)n2)nc1] 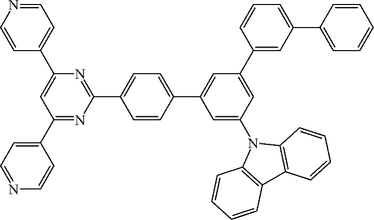 [SMILES: c1ccc(-c2cccc(-c3cc(-c4ccc(-c5nc(-c6ccncc6)cc(-c6ccncc6)n5)cc4)cc(-n4c5ccccc5c5ccccc54)c3)c2)cc1] 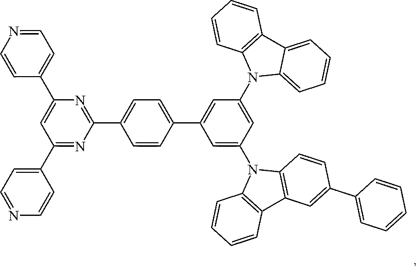 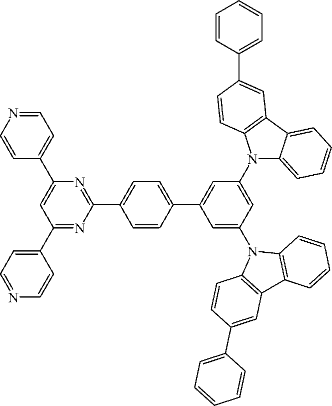 [SMILES: c1ccc(-c2ccc3c(c2)c2ccccc2n3-c2cc(-c3ccc(-c4nc(-c5ccncc5)cc(-c5ccncc5)n4)cc3)cc(-n3c4ccccc4c4cc(-c5ccccc5)ccc43)c2)cc1] 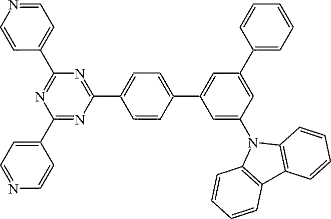 [SMILES: c1ccc(-c2cc(-c3ccc(-c4nc(-c5ccncc5)nc(-c5ccncc5)n4)cc3)cc(-n3c4ccccc4c4ccccc43)c2)cc1] 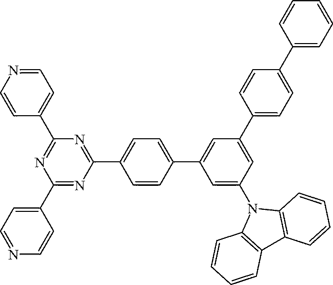 [SMILES: c1ccc(-c2ccc(-c3cc(-c4ccc(-c5nc(-c6ccncc6)nc(-c6ccncc6)n5)cc4)cc(-n4c5ccccc5c5ccccc54)c3)cc2)cc1] 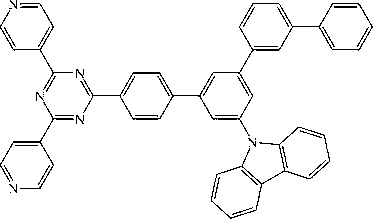 [SMILES: c1ccc(-c2cccc(-c3cc(-c4ccc(-c5nc(-c6ccncc6)nc(-c6ccncc6)n5)cc4)cc(-n4c5ccccc5c5ccccc54)c3)c2)cc1] 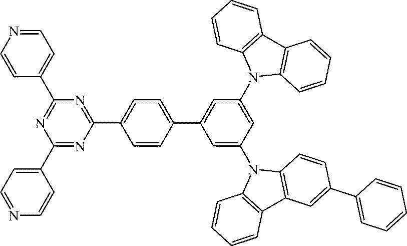 [SMILES: c1ccc(-c2ccc3c(c2)c2ccccc2n3-c2cc(-c3ccc(-c4nc(-c5ccncc5)nc(-c5ccncc5)n4)cc3)cc(-n3c4ccccc4c4ccccc43)c2)cc1] 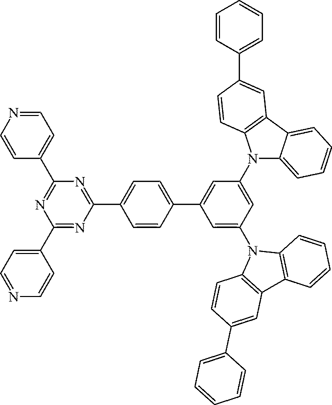 [SMILES: c1ccc(-c2ccc3c(c2)c2ccccc2n3-c2cc(-c3ccc(-c4nc(-c5ccncc5)nc(-c5ccncc5)n4)cc3)cc(-n3c4ccccc4c4cc(-c5ccccc5)ccc43)c2)cc1] 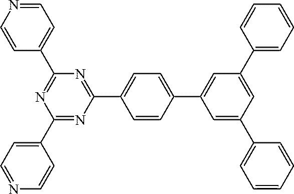 [SMILES: c1ccc(-c2cc(-c3ccccc3)cc(-c3ccc(-c4nc(-c5ccncc5)nc(-c5ccncc5)n4)cc3)c2)cc1] 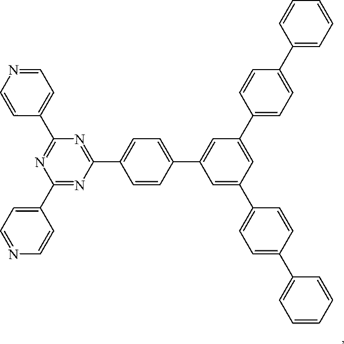 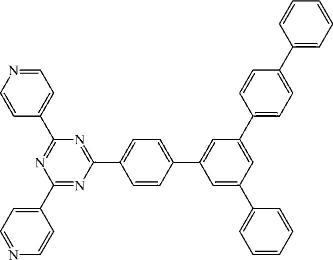 [SMILES: c1ccc(-c2ccc(-c3cc(-c4ccccc4)cc(-c4ccc(-c5nc(-c6ccncc6)nc(-c6ccncc6)n5)cc4)c3)cc2)cc1] 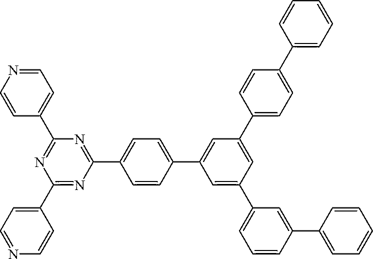 [SMILES: c1ccc(-c2ccc(-c3cc(-c4ccc(-c5nc(-c6ccncc6)nc(-c6ccncc6)n5)cc4)cc(-c4cccc(-c5ccccc5)c4)c3)cc2)cc1] 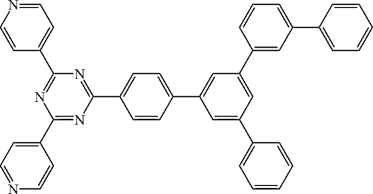 [SMILES: c1ccc(-c2cccc(-c3cc(-c4ccccc4)cc(-c4ccc(-c5nc(-c6ccncc6)nc(-c6ccncc6)n5)cc4)c3)c2)cc1] 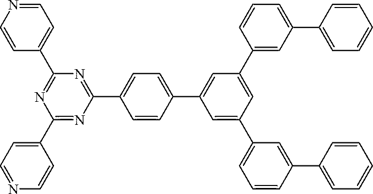 [SMILES: c1ccc(-c2cccc(-c3cc(-c4ccc(-c5nc(-c6ccncc6)nc(-c6ccncc6)n5)cc4)cc(-c4cccc(-c5ccccc5)c4)c3)c2)cc1] 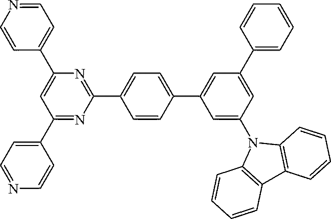 [SMILES: c1ccc(-c2cc(-c3ccc(-c4nc(-c5ccncc5)cc(-c5ccncc5)n4)cc3)cc(-n3c4ccccc4c4ccccc43)c2)cc1] 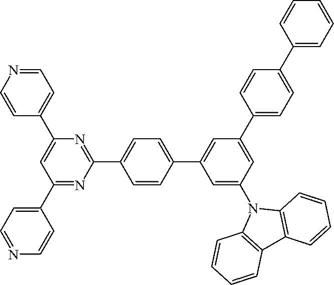 [SMILES: c1ccc(-c2ccc(-c3cc(-c4ccc(-c5nc(-c6ccncc6)cc(-c6ccncc6)n5)cc4)cc(-n4c5ccccc5c5ccccc54)c3)cc2)cc1] 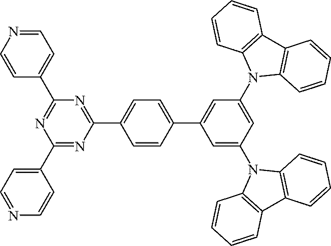 [SMILES: c1ccc2c(c1)c1ccccc1n2-c1cc(-c2ccc(-c3nc(-c4ccncc4)nc(-c4ccncc4)n3)cc2)cc(-n2c3ccccc3c3ccccc32)c1] 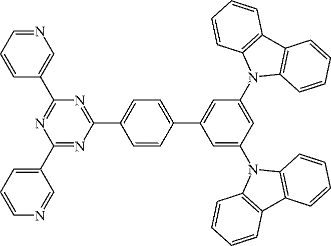 [SMILES: c1cncc(-c2nc(-c3ccc(-c4cc(-n5c6ccccc6c6ccccc65)cc(-n5c6ccccc6c6ccccc65)c4)cc3)nc(-c3cccnc3)n2)c1] 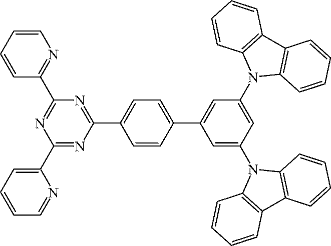 [SMILES: c1ccc(-c2nc(-c3ccc(-c4cc(-n5c6ccccc6c6ccccc65)cc(-n5c6ccccc6c6ccccc65)c4)cc3)nc(-c3ccccn3)n2)nc1] 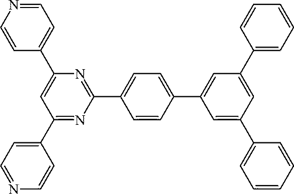 [SMILES: c1ccc(-c2cc(-c3ccccc3)cc(-c3ccc(-c4nc(-c5ccncc5)cc(-c5ccncc5)n4)cc3)c2)cc1] 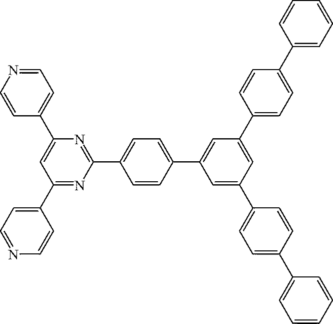 [SMILES: c1ccc(-c2ccc(-c3cc(-c4ccc(-c5ccccc5)cc4)cc(-c4ccc(-c5nc(-c6ccncc6)cc(-c6ccncc6)n5)cc4)c3)cc2)cc1] 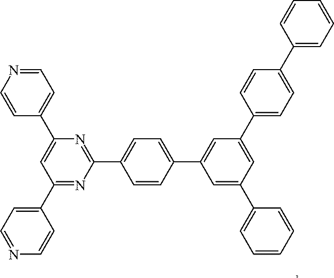 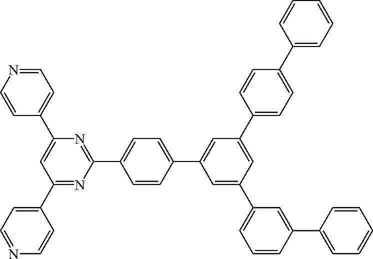 [SMILES: c1ccc(-c2ccc(-c3cc(-c4ccc(-c5nc(-c6ccncc6)cc(-c6ccncc6)n5)cc4)cc(-c4cccc(-c5ccccc5)c4)c3)cc2)cc1] 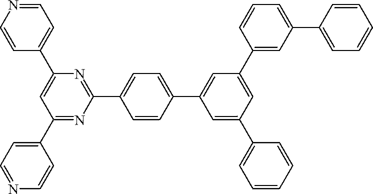 [SMILES: c1ccc(-c2cccc(-c3cc(-c4ccccc4)cc(-c4ccc(-c5nc(-c6ccncc6)cc(-c6ccncc6)n5)cc4)c3)c2)cc1] 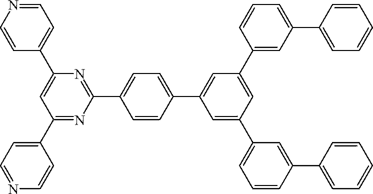 [SMILES: c1ccc(-c2cccc(-c3cc(-c4ccc(-c5nc(-c6ccncc6)cc(-c6ccncc6)n5)cc4)cc(-c4cccc(-c5ccccc5)c4)c3)c2)cc1] 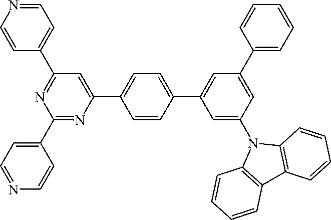 [SMILES: c1ccc(-c2cc(-c3ccc(-c4cc(-c5ccncc5)nc(-c5ccncc5)n4)cc3)cc(-n3c4ccccc4c4ccccc43)c2)cc1] 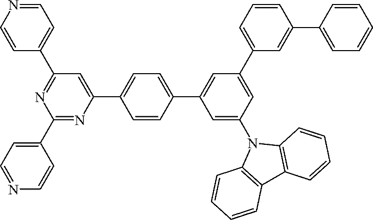 [SMILES: c1ccc(-c2cccc(-c3cc(-c4ccc(-c5cc(-c6ccncc6)nc(-c6ccncc6)n5)cc4)cc(-n4c5ccccc5c5ccccc54)c3)c2)cc1] 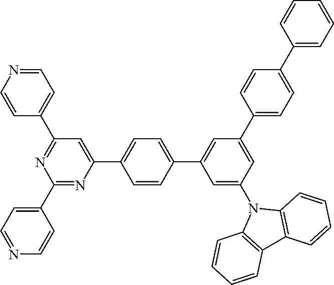 [SMILES: c1ccc(-c2ccc(-c3cc(-c4ccc(-c5cc(-c6ccncc6)nc(-c6ccncc6)n5)cc4)cc(-n4c5ccccc5c5ccccc54)c3)cc2)cc1] 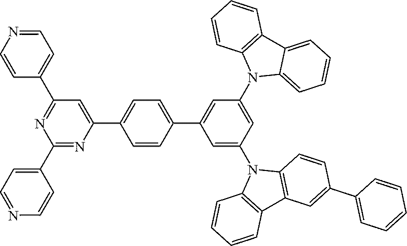 [SMILES: c1ccc(-c2ccc3c(c2)c2ccccc2n3-c2cc(-c3ccc(-c4cc(-c5ccncc5)nc(-c5ccncc5)n4)cc3)cc(-n3c4ccccc4c4ccccc43)c2)cc1] 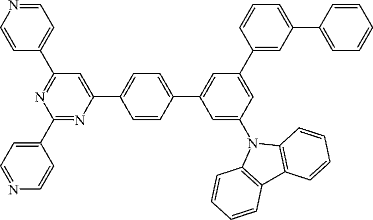 [SMILES: c1ccc(-c2cccc(-c3cc(-c4ccc(-c5cc(-c6ccncc6)nc(-c6ccncc6)n5)cc4)cc(-n4c5ccccc5c5ccccc54)c3)c2)cc1] 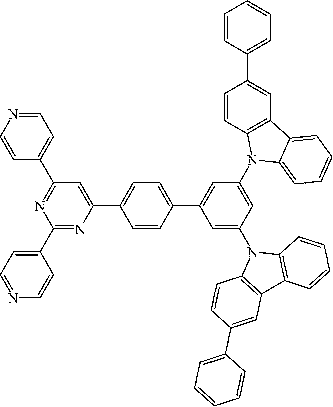 [SMILES: c1ccc(-c2ccc3c(c2)c2ccccc2n3-c2cc(-c3ccc(-c4cc(-c5ccncc5)nc(-c5ccncc5)n4)cc3)cc(-n3c4ccccc4c4cc(-c5ccccc5)ccc43)c2)cc1] 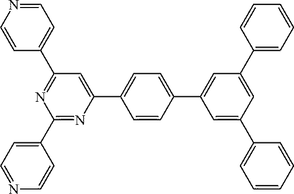 [SMILES: c1ccc(-c2cc(-c3ccccc3)cc(-c3ccc(-c4cc(-c5ccncc5)nc(-c5ccncc5)n4)cc3)c2)cc1] 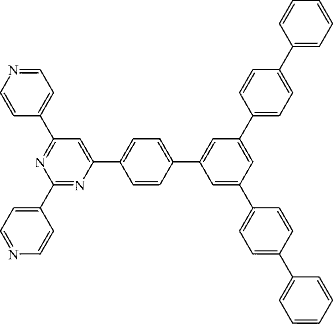 [SMILES: c1ccc(-c2ccc(-c3cc(-c4ccc(-c5ccccc5)cc4)cc(-c4ccc(-c5cc(-c6ccncc6)nc(-c6ccncc6)n5)cc4)c3)cc2)cc1] 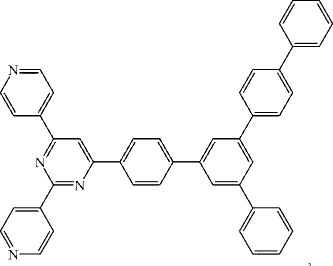 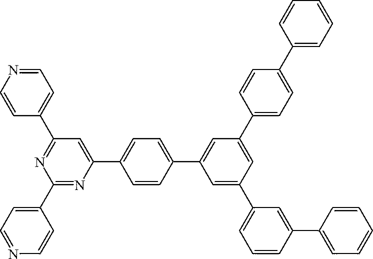 [SMILES: c1ccc(-c2ccc(-c3cc(-c4ccc(-c5cc(-c6ccncc6)nc(-c6ccncc6)n5)cc4)cc(-c4cccc(-c5ccccc5)c4)c3)cc2)cc1] 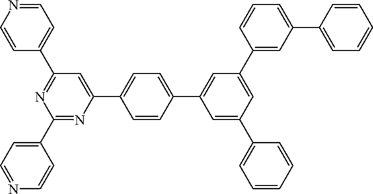 [SMILES: c1ccc(-c2cccc(-c3cc(-c4ccccc4)cc(-c4ccc(-c5cc(-c6ccncc6)nc(-c6ccncc6)n5)cc4)c3)c2)cc1] 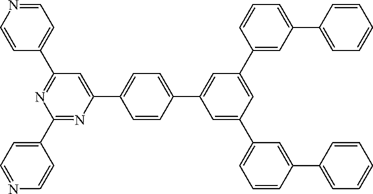 [SMILES: c1ccc(-c2cccc(-c3cc(-c4ccc(-c5cc(-c6ccncc6)nc(-c6ccncc6)n5)cc4)cc(-c4cccc(-c5ccccc5)c4)c3)c2)cc1] 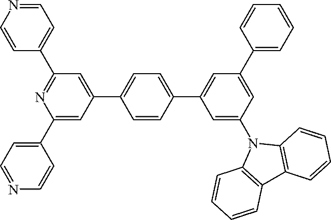 [SMILES: c1ccc(-c2cc(-c3ccc(-c4cc(-c5ccncc5)nc(-c5ccncc5)c4)cc3)cc(-n3c4ccccc4c4ccccc43)c2)cc1] 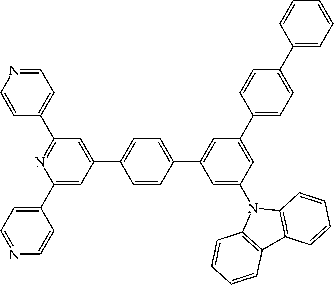 [SMILES: c1ccc(-c2ccc(-c3cc(-c4ccc(-c5cc(-c6ccncc6)nc(-c6ccncc6)c5)cc4)cc(-n4c5ccccc5c5ccccc54)c3)cc2)cc1] 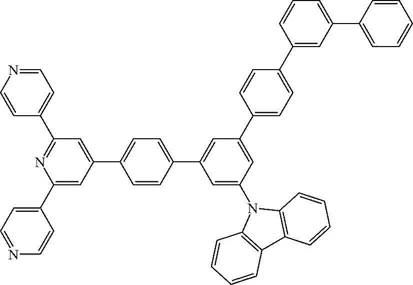 [SMILES: c1ccc(-c2cccc(-c3ccc(-c4cc(-c5ccc(-c6cc(-c7ccncc7)nc(-c7ccncc7)c6)cc5)cc(-n5c6ccccc6c6ccccc65)c4)cc3)c2)cc1] 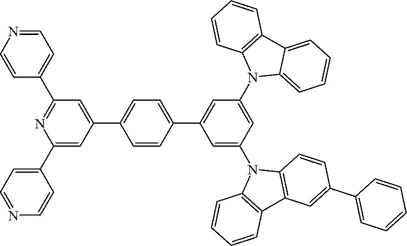 [SMILES: c1ccc(-c2ccc3c(c2)c2ccccc2n3-c2cc(-c3ccc(-c4cc(-c5ccncc5)nc(-c5ccncc5)c4)cc3)cc(-n3c4ccccc4c4ccccc43)c2)cc1] 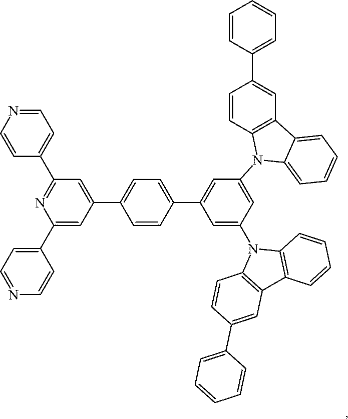 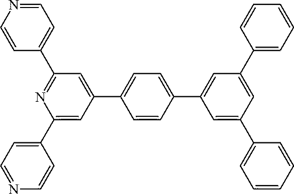 [SMILES: c1ccc(-c2cc(-c3ccccc3)cc(-c3ccc(-c4cc(-c5ccncc5)nc(-c5ccncc5)c4)cc3)c2)cc1] 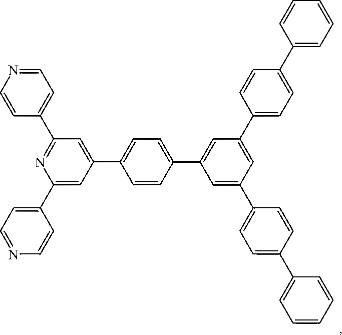 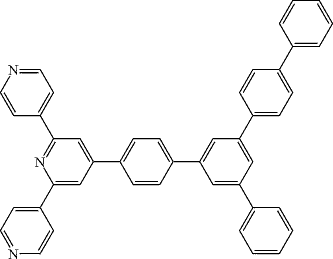 [SMILES: c1ccc(-c2ccc(-c3cc(-c4ccccc4)cc(-c4ccc(-c5cc(-c6ccncc6)nc(-c6ccncc6)c5)cc4)c3)cc2)cc1] 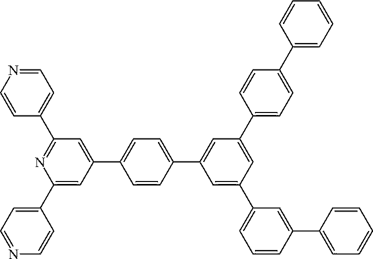 [SMILES: c1ccc(-c2ccc(-c3cc(-c4ccc(-c5cc(-c6ccncc6)nc(-c6ccncc6)c5)cc4)cc(-c4cccc(-c5ccccc5)c4)c3)cc2)cc1] 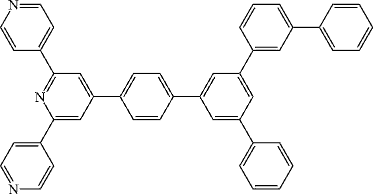 [SMILES: c1ccc(-c2cccc(-c3cc(-c4ccccc4)cc(-c4ccc(-c5cc(-c6ccncc6)nc(-c6ccncc6)c5)cc4)c3)c2)cc1] 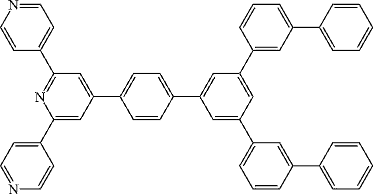 [SMILES: c1ccc(-c2cccc(-c3cc(-c4ccc(-c5cc(-c6ccncc6)nc(-c6ccncc6)c5)cc4)cc(-c4cccc(-c5ccccc5)c4)c3)c2)cc1]
.
